(19) United States
(12) Patent Application Publication
(10) Pub. No.: US 2006/0188870 A1
(43) Pub. Date: Aug. 24, 2006

(54) Identification of active sites in enzymes

(76) Inventors: Avi Ben-Shimon, Rechovot (IL); Miriam Eisenstein, Rechovot (IL)

Correspondence Address: Martin D. Moynihan;PRTSI, Inc., P.O. Box 16446, Arlington, VA, 22215, US

(21) Appl. No.: 11/359,514

(22) Filed: Feb. 23, 2006

Related U.S. Application Data
(60) Provisional application No. 60/655,421, filed on Feb. 24, 2005.

(51) Int. Cl. C12Q 1/00; G06F 19/00

(52) U.S. Cl. 435/4; 702/19

(57) ABSTRACT

A method for determining the location of the active site of an enzyme is provided. The method comprising: determining a location of a reference point inside a functional unit of the enzyme; determining a limiting distance from the reference point; and identifying one or more molecular surface portions within the limiting distance to determine whether the molecular surface portion is part of the active site.

### RELATED APPLICATIONS

This Application claims priority from U.S. Provisional Patent Application No. 60/655,421, filed on Feb. 24, 2005, the contents of which are incorporated herein by reference.

### FIELD OF THE INVENTION

The present invention is of a system and method for correctly predicting the location of catalytic site(s) of enzymes through dry, in silico analysis.

### BACKGROUND OF THE INVENTION

Biological research increasingly focuses on proteomics in the current, post-genomic era. Experimental and computational efforts are devoted to large-scale analyses of information derived from the 3D structures of proteins with the goal of understanding the processes in the living cell and for drug design and discovery [1, 2]. The three dimensional structures of protein targets are preferred starting points for drug design projects [3-6]. After solving the structure, the next essential step is the extraction of the function from the structure. The conventional experimental procedures are often time and money consuming, therefore the importance of developing new in-silico methods in order to locate potential functional sites in novel enzymes with unknown function is clear; especially as the number of solved yet unannotated structures increases by the contribution of various structural genomics initiatives [7].

Various methods have been developed in order to characterized and detect functional sites in proteins. Some methods use evolutionary conservation data combined with structural information [8-11], other infer the function by comparison to known global [12, 13] or local [14-17] folds. Recently a detailed and comprehensive analysis of a large dataset of enzymes showed that catalytic residues have several common characteristics, including solvent accessibility, secondary structure type, residue type and evolutionary conservation [18]. These properties together with additional descriptors were used in a neural network and successfully identified 69% of the active site residues and further 25% were partially predicted [19].

In many cases a more difficult and complex problem must be addressed: extraction of the function in cases where neither evolutionary nor structural similarity data are available, relying just on the 3D structure of the molecule. The methods which relay solely on the 3D structure are usually geometrical, but functional residues were also identified by computing structure energetics and ionization properties [20, 21].

Enzymes active sites and binding sites are usually characterized by large and deep clefts [22-25]. This phenomenon is probably a consequence of the physical principles that govern molecular recognition. On the one hand, high affinity can be gained by sufficiently large interaction surface. On the other hand, specificity is more easily attained in locations that impose strict geometric and chemical constrains. On this basis, purely geometrical computational tools have been developed to detect potential active or binding sites [25-30]. Although the different methods exploit the same phenomenon, i.e. searching for “large clefts”, each uses a different algorithm. The currently main approaches are surveyed in the following:

1. The global and detailed layer approach. This is the most common approach in which a global envelope surface (molecular sea level) is compared too a more detailed representation of the molecule. The differences between the two layers are used for cavity identification. Different methods use different approaches for determining the global and detailed layers of the molecule. The early and simple methods used spheres or ellipsoids to define the protein “sea level” [31, 32]. Others compute the solvent accessible or molecular surface using different probe sizes [30]. The more recent approaches like APROPOS and CAST use the alpha shape theory. APROPOS [27] detects protein depressions by comparing surfaces which were generated using two different alpha values. An envelope surface with a large alpha value describes the global shape whereas a smaller alpha value creates the detailed surface layer. The CAST program [25, 33] also uses the alpha shape theory in order to determine the molecular shape, but it defines pockets using the discrete flow method.

2. The grid based approach (like in the program LIGSITE which is based on POCKET [26, 29]) in which the protein is embedded in a regularly spaced grid where each accessible grid point is scored by its degree of burial in surface depressions. The degree of burial is determined by scanning the grid lines along the three Cartesian axes and the four cubic diagonals for areas that are enclosed by protein atoms on both sides. Adjacent points with high burial values are then clustered to form cavities.

3. The probe based approach, for example the PASS program. The protein is coated by a layer of spherical probes. Probes which clash with the protein, are not sufficiently buried or are located too close to a more buried probe are filtered out. A new layer of probes is than constructed on previously recognized probes and the filtering is repeated. The process is continued until no probes in a new layer survive the filter. Finally, the putative site is determined by identifying the probe with the highest burial degree and the largest number of probes in its vicinity [28]. Another method using probes is the solvent mapping approach. Different small organic molecules are docked on the protein surface. The consensus active site is determined by the number of different probes that bind to it [34].

Very recently a new algorithm based on molecular dynamics simulations was developed for detecting and quantifying cavities and surface grooves. It is based on the observation that the mobility of water molecules in cavities is significantly lower than that of the bulk water [35].

All the geometrical methods search, define and rank protein pockets/cavities. The different sea level definitions, and in consequence the differences in determining the cavity outer border, as well as the question of what should be considered a cavity, are the source of considerable differences in the performance of the different algorithms. The following example emphasizes the problem: Laskowski et al, using the SURFNET program reported on a correlation between the enzyme size and the volume of the largest cleft, although there were considerable variations [23]. Using the same dataset of structures and the CAST program Liang et al found no such correlation [25].

In general, when the active site is large most of the programs are able to recognize it. However, when a large number of cavities is found for a given enzyme and especially when there is no one dominant cavity, the determination of the correct active site is more difficult and the differences between the algorithms manifest themselves. Therefore, finding a new reference point for looking at surface cavities, which is independent of the protein sea level or of the cavity definition to discriminate and rank enzyme surface depressions, would be of great value.

Examination of several well established properties of active sites and catalytic residues implies that enzymes functional sites should be positioned close to the enzyme center of mass: (i) Active sites are very often a cavity, which allows the enzymatic reaction to be preformed away from the surrounding solvent [22-25]. In order to create a cavity, mass must be built around it. (ii) Catalytic residues tend to have lower B-factor values than other residues; indeed B-factor values are known to be correlated to the distance from the protein centroid [36, 37]. (iii) Catalytic residues are partially buried and often involved in hydrogen bonds, hence their mobility is further restricted [18], yet the residues near the outer edge of the active site cavity (mainly loop regions) are mobile in order to attain recognition [38]. (iv) Recently catalytic residues were found to be central network nodes, therefore they can affect or be affected by most of the other surrounding residues [39].

### SUMMARY OF THE INVENTION

The present invention provides a method and system for predicting the location of catalytic site(s) of enzymes through “dry”, in silico analysis. Through analysis of the structures of a large dataset of enzymes, the present inventors discovered that the catalytic residues and therefore active sites of enzymes are found in close proximity to the enzyme's centroid. This trend is observed when the positions of all the Cα atoms of the enzyme, including those of the core, are compared to the catalytic residues' Cα atoms, indicating that this property is embedded in the enzyme's fold. When considering only the surface area of the enzymes, in 80% of the enzymes at least one catalytic residue is included among the 5% surface residues closest to the enzyme's centroid.

In contrast to currently known methods, which search and define all of the cavities/pockets on an enzyme's surface, the method of the present invention searches for a limited number of depressions in the surface or for internal voids, which are close to a fixed reference point, which is inside the molecule, for example the centroid of the molecule. This choice of reference point eliminates the need to define a protein sea level.

According to a preferred embodiment of the present invention, the method is implemented in software, which may also optionally include firmware or instructions executed through hardware execution. A non-limiting example of such an implementation was provided in a new algorithm for prediction of enzyme active site location, named EnSite. EnSite was applied to two datasets in an experiment which clearly demonstrated the accuracy of the method and system of the present invention. In a monomeric dataset of 65 enzymes the algorithm correctly predicted the active site location in 97% of the cases. In a more comprehensive dataset of 176 enzymes, the predictions were correct in 86% of the cases. Moreover, it is shown that the principle of closeness of the active site to the centroid of the molecule is firm when the active subunit of the protein is correctly defined. The active subunit can be a single monomer, chain or domain or a group of monomers, chains or domains that together form the functional unit. The new, centroid-active site proximity property was also implemented in a post scan filter that re-ranked enzyme-inhibitor docking results. Docking is the prediction of the structures of molecular complexes starting from the structures of the uncomplexed (unbound) component molecules. All the relative translations and rotations of the two molecules are tested in a docking scan and for each position the geometric and chemical complementarity (“score”) between the two molecules is evaluated. The scan produces a very large number of docking solutions which are ranked by their scores. If the function used for calculating the score is good then the correct structure of the complex obtains the best score (or one of the best scores). Additional physical and biological information may optionally be used to re-evaluate and re-rank the docking solutions. The centroid-active site distance filter (see Methods), one embodiment of the present invention, was applied to the unbound docking results obtained for 9 complexes using the docking program MolFit, also by the present group [57-64]. This filter produced an exceptional improvement in the ranks of the nearly correct docking predictions.

### BRIEF DESCRIPTION OF THE DRAWINGS

The invention is herein described, by way of example only, with reference to the accompanying drawings, wherein:

FIG. 1a shows the distribution of catalytic residues according to their CAPC and SAPC values, which measure their distances from the molecular centroid relative to other residues (see Methods). Note the large proportion of catalytic residues which are in close proximity to the molecular centroid compared to the rest of the enzyme. This proportion increases markedly when only the surface residues are considered (SAPC values);

FIG. 1b shows analyses of the cumulative fractions of enzymes or catalytic residues with given SAPC or CAPC values (see Method for definition) for the dataset of Bartlett et al (dataset-II). Each enzyme is represented by the lowest SAPC or CAPC value for any of the catalytic residues. Note that for 80% of the enzymes in the dataset, at least one catalytic residue that contributes to the enzyme's surface is found among the 5% surface residues that are closest to the enzyme's centroid. The proportion increases to 94% when 15% of the surface residues closest to the centroid are considered;

FIG. 2 shows the 1^{st} sphere internal clusters distribution in dataset-I. Only the largest internal voids were included in the list of predicted active sites. A p-value of 0.05 was selected as the limit, corresponding to 750 surface dots (Mcluster);

FIGS. 3a-3d show whole protein analysis as compared to separate domain analysis for 1PII and 1BLL;

FIG. 4 shows the deviation of the percent of correct predictions ranked 1 or NF in different biological structural groups (monomers, homo-multimers, hetero-multimers) from the distribution of enzymes with different biological units in dataset-II;

FIGS. 5a-5b show examples of enzymes in which the catalytic residues reside in two different chains;

FIGS. 6a-6b show separate and combined domain analysis in cases in which the catalytic residues (depicted in green) are located on more than one domain;

FIG. 7 shows catalytic atoms recognition in domain analysis, in cases in which the catalytic residues are located in two different domains;

FIG. 8 shows the prediction of the active site in the chemotaxis receptor methylesterase (1A2O), in which the functional unit consists of both the C and N terminal domains;

FIGS. 9a-9c show examples of active sites formed by more than one chain although the catalytic residues (shown in green) are located on a single chain;

FIGS. 10a-10c show examples of shallow/flat active sites;

FIGS. 11a-11b show examples of buried active sites;

FIG. 12 shows the dependence of the rank of the correctly predicted site and the ratio of enzyme surface area to pocket area for 5 enzymes with buried active sites;

FIG. 13 shows the correlation between the closeness centrality values computed with the SARIG server and the average distance from enzyme centroid for the 662 amino acids of 1FOH;

FIG. 14 shows an outline of the active site prediction algorithm as implemented in the software program EnSite;

FIG. 15 shows how is the parameter Cdmax determined for a given surface dot density; and

FIGS. 16a-16d show a schematic illustration of the main concepts in the active site prediction algorithm EnSite.

### DESCRIPTION OF THE PREFERRED EMBODIMENTS

The present invention provides (i) a method for predicting the location of the active site in an enzyme through dry, in silico analysis, (ii) a method to determine the functional unit of an enzyme; (iii) a method to re-evaluate enzyme-ligand docking solutions. Preferably, the method (i) features determining the location of the active site by identifying portions of the surface of the enzyme which are closest to the centroid of the enzyme. These portions are then ranked by their size. Therefore, the location of the enzyme active site is preferably determined according to relative distance from the centroid, rather than by examining features on the surface of the enzyme itself. The method of determining the functional unit of an enzyme features determining the location of fragments of the active site on different monomers, domains or chains and identification of the functional unit as the assembly of monomers, domains or chains which contain spatially continuous active site fragments. The method of re-evaluating enzyme-ligand docking solutions features determining the structure of enzyme-ligand complexes by re-ranking a large number of docking solutions by the distance between the enzyme-ligand interface and the enzyme centroid.

According to a preferred embodiment of the present invention, if one or more residues of the active site or interaction site are known through another method (for example through site directed mutagenesis), then optionally and preferably the present invention may be used to verify the three dimensional model of the enzyme, at least with regard to its accuracy in terms of the location of the active site on the surface of the enzyme.

According to another preferred embodiment of the present invention, after the location of the active site or interaction site is determined as a portion of the surface of the enzyme or ligand, optionally and preferably one or more residues of the active site may be located through an additional process. For example, site directed mutagenesis may optionally be used to locate one or more residues. Alternatively, one or more additional modeling algorithms, as those described in the Background section, may be used to locate specific active site residues.

The results obtained in the SAPC and CAPC analyses (and described in greater detail below with regard to the Methods and Examples) showed that in most enzymes the catalytic residues are located near the centroid of the molecule. This property is used in a new algorithm designed to identify enzyme active sites (EnSite), which produces a very high extent of prediction success. Moreover, using all the domains and chains which are directly involved in catalysis further improved the prediction results, indicating that the close proximity of active sites to the centroid is a property of the functional unit. It is also shown that using the new property for re-ranking of docking solutions exceptionally improves the rank of the correct solution.

In contrast to other geometrical methods for active site prediction which search and define cavities/pockets on the enzyme's surface, the method of the present invention adopts a different point of view: it “looks from the inside out” and searches only for a limited number of surface portions which reside in close proximity to the centroid of the molecule. In addition to the high extent of prediction success, our unique approach proves to be particularly effective in the non trivial cases, such as buried or flat and shallow active sites. Although EnSite ranks the surface clusters by their size, in more than 90% of the cases in which the largest cluster represents the correct active site, it is also closest to the enzyme's centroid. This result indicates that the active site cavities are very often the closest surface portions to the molecular centroid (the results are described in greater detail with regard to the Examples below).

An interesting and fundamental question which this study brings up is why are catalytic residues and active sites of enzymes located in close proximity to the molecular centroid?

Without wishing to be limited by a single hypothesis, the reason may be that positioning the active site near the center of the molecule supports several active sites characteristic which are essential for their functionality.

Recently, it was shown that high closeness centrality values characterize catalytic and functional residues, suggesting that these residues are in an optimal position to effectively disseminate and receive information from the rest of the protein [39].

Ligands usually interact with only a small number of residues, however, their effect often propagate to other portions of the protein. This propagation may be limited to regions immediately adjacent to binding or may extend to distal regions [65-69]. Positioning of the active site near the centroid of the enzyme contributes to the catalytic residues centrality as we showed by the high correlation between closeness centrality values and the distance to the enzyme centroid (R in the range 0.94-0.96), resulting in short path for propagating the signal to different parts of the enzymes.

Luque et al analyzed 16 non-structurally homologues proteins (11 enzymes) and showed that binding sites are characterized by the presence of both low and high stability regions. In most cases the low stability regions were loops that become stable upon ligand binding. However for most of the enzymes the residues directly implicated in catalysis were located in the most stable regions of the binding site [38]. The authors suggested that this is important for catalytic efficiency which requires a well defined stereochemical arrangement of the participating groups that therefore should be held in place.

Bartlett et al reported on a related trend; they found that catalytic residues tend to have lower B-factors than other residues [18]. Crystallographic B-factors are a measure of the thermal fluctuations of atoms, groups, or molecules, and their analysis provides information on the mobility of the protein. In crystallographic refinement each atom is assigned a B-factor which is proportional to the mean square amplitude of the motion. The thermal fluctuations arise from internal modes and external modes. The internal modes represent thermal deformations of the molecule whereas the external modes describe motion of the whole molecule as a rigid-body (this motion was described by the Translation-Liberation-Screw (TLS) model [37]). A simple model in which B-factors were calculated assuming that they are proportional to the square of the distance of the atoms from the protein's centroid, showed moderate correlation with the experimental B-factors [36] (average correlation coefficient of 0.515). We calculated the correlation coefficient between the B-factors and the square of the distance from the molecular centroid for all enzymes in datasets 1 and 11, and found that monomers tend to have higher correlations coefficients than multimeric enzymes. Thus an average correlation coefficient of R=0.57 was obtained for the entire monomeric dataset-I. For the single domain monomers in this dataset the average coefficient was higher than for multi domain monomers, R=0.6 and R=0.56, respectively. Similarly the average correlation coefficient for the monomeric enzymes in dataset-II is much higher than for the rest of the enzymes, R=0.59 versus R=0.51.

Therefore, thermal mobility of catalytic residues can be lowered by placing them near the center of the molecule. Additional structural stabilization is achieved by partial buried of the catalytic residues and by formation of hydrogen bonds [18].

Again without wishing to be limited by a single hypothesis, the active site may be so located in order for the mass to be distributed around the functional site in order to maintain it.

Jones and Thornton have recently reviewed the complex relationship between protein fold and function, highlighting the necessity of looking beyond the global fold of a protein to specific sites within them [70]. This complex relationship is reflected by the observation that proteins with the same fold can perform many diverse biological functions [71, 72]. For instance, the triose phosphate isomerase (TIM) barrel fold is associated with 61 different EC numbers [73], covering five of the six top level EC classifications [74]. Conversely, the same function can be achieved by more than one protein fold [15]. Another validation to this claim, comes from several protein engineering studies, in which a well ordered functional site was successfully transferred to a different structural scaffold [75-80].

If so, what is the function of the bulk of the enzyme that does not constitute the active site? The view that Haldane first offered [81] and was later popularized by Pauling [82, 83] that the bulk of the enzyme exists for the purpose of maintaining the active site in geometry faithful to the transition state structure of the reaction the enzyme has evolved to catalyze. Whether it is the transition state or the ground state like proposed by the Shifting Specificity Model (SSM) [84] is an open debate [85]. Obviously, the active site spatial arrangement can not exist by it self, it needs a stable scaffold to create it and maintain it. Hence, it seems that the mass around the desired function is created in order to achieve the optimal active site spatial arrangement, but the global fold is less important as long as this spatial arrangement is achieved.

Most active sites are located within deep clefts. Clefts provide an optimal environment for a catalytic reaction by separating it from the surrounding solvent. They supply large interaction surface with the ligand and may provide ligand specificity control. In order to create a cleft, mass must be built around it. The following example emphasizes how evolution uses the notion of “mass around the function” in order to exploit the same catalytic machinery but allow for different ligand specificities by modifying the mass around the functional site. The serum paraoxonase (PON) family has one of the broadest specificities known. Until recently it was unclear how PON exhibits such a broad spectrum of activities and what dictates the substrate selectivity. Using directed evolution experiments it became possible to follow the footsteps of natural evolution, generating new PON variants with predetermined specificities [86]. The newly evolved variants clearly defined a limited set of amino acids whose alteration markedly shifts reactivity and substrate selectivity. Notably, these amino acids delineate the entrance to the active site and its “walls” and thus govern substrate selectivity. The authors suggested that the PON subfamilies diverged during evolution but maintained their overall active site structure and catalytic machinery. Yet, their substrate and reaction selectivities changed markedly [86].

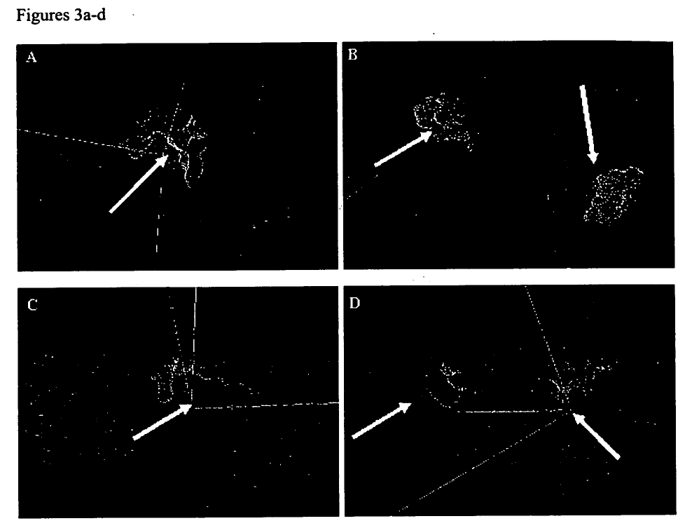

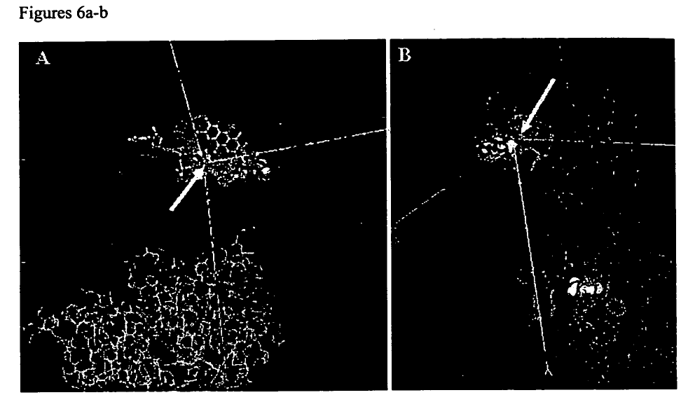

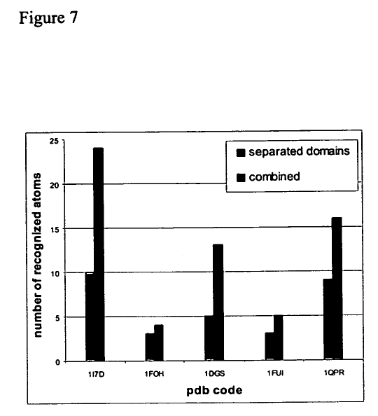

Based on the concept of “mass around function”, and again without wishing to be limited by a single hypothesis, it may be that for a simple theoretical enzyme in which the catalytic function is the only function, the fold of the polypeptide chain evolved in such a way that its mass is distributed around the functional site. The functional residues in this case, will be positioned close to the enzyme center of mass (or centroid). The notion of “mass around function” is supported by our results. Thus, the close proximity of active sites to the molecular centroid is more conspicuous when the functional unit of the enzyme is considered, namely the unit which includes the catalytic residues or which participates in forming the active site (see Tables 4-6 & FIGS. 3, 5, 6, and 9 below). When the catalytic residues are positioned on more than one chain/domain, application of EnSite to each chain/domain separately usually, but not always, identified the relevant catalytic residues; yet recognition usually improved when the chains/domains were combined to form the functional unit of the enzyme (FIG. 7, table 4). Possibly a part of the combined function existed on one or more of the domains/chains before another domain/chain join it, hinting at step wise evolution of such enzymes (FIG. 6).

Another related result is the anti-correlation between the prediction performance of EnSite and the biological complexity of the enzyme; EnSite is most successful for monomeric enzymes and least successful for hetero-multimeric enzymes. A single domain monomeric enzyme is close to the theoretical enzyme which carries only one functional site, because it is less likely to have additional functional sites, such as recognition sites for other monomers or subunits. In contrast, a highly complex multimeric enzyme largely deviates from this definition because it must have additional functional sites for creating the complex structure. Interestingly, in several cases in which EnSite failed to predict the correct active site, the proposed 1^{st} cluster represented the interface with another chain.

### Methods

The following methods and data were used to examine the operation of the method of the present invention, as implemented in a non-limiting example as the EnSite software program. Results of the subsequent analysis are described with regard to Examples 1-5 below.

Datasets

Two datasets of enzymes with known structures were used in this study:

Dataset-I includes 65 monomers out of the 67 in the dataset of Laskowski et al [23]. We excluded 2 proteins, which are not enzymes: the integral membrane porin 2POR and the bacteriochlorophyll-A protein 3BCL. This dataset comprises only monomeric enzymes making it particularly adequate for testing our active site prediction algorithm (see below). Moreover, it was previously used by several groups and can therefore serve for comparing the performance of our prediction algorithm with other algorithms.

Dataset-II includes 176 enzymes out of the 177 hand annotated enzymes reported in the catalytic site atlas version 1.0 [46], based on the study of Bartlett et al [18, 46]. At the time of this study only 176 of the 177 enzymes were available for download. This representative dataset covers all six top level enzyme classifications [73]. It was manually annotated to include an accurate list of the catalytic residues in each enzyme. For 172 of the 176 enzymes in this dataset we used only a single chains in our prediction procedure, the chain that includes the catalytic residues. For the other 4 structures (1MHL, 1BCR, 1AHJ, 1PYA), in which the catalytic residues are found on more than one non identical chain, we considered only the largest chain in these 4 cases.

Notably, only 12 enzymes are common to the two datasets, 3 enzymes which have the same PDB code and 9 enzymes with different PDB codes but with the same E.C. number.

Analysis of the proximity of catalytic residues to the enzyme centroid

This analysis was applied to the 176 enzymes in dataset II. For each catalytic residue the following two values were calculated:

1. The Surface Atoms Proximity to the Centroid (SAPC) is calculated as follow: First, the distance from the protein centroid to each atom that contributes to the molecular surface is calculated, as described in greater detail below. Next, the shortest distance for each catalytic residue is ranked relatively to all other distances in a scale of 0-100%.

2. The Cα Atoms Proximity to the Centroid (CAPC) parameter is calculated in a similar manner to the SAPC parameter but it considers the distance of all the Cα atoms from the molecular centroid. Thus, it compares the distance of the Cα atom in each catalytic residue to the distribution of distances for all the Cα atoms in the enzyme, on a scale of 0-100%.

Prediction of the Active Site—the EnSite Algorithm

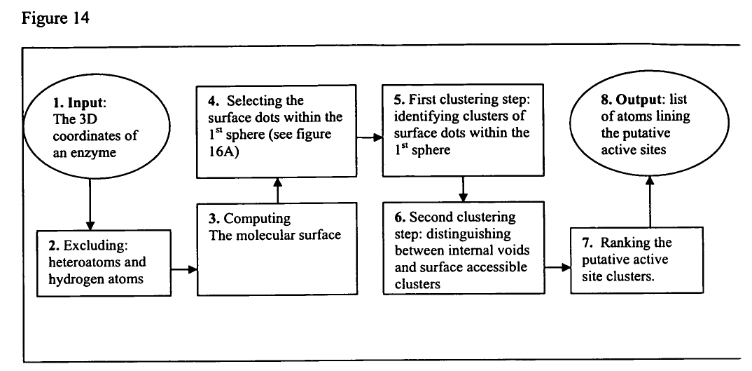

FIG. 14 shows the active site prediction algorithm as implemented in the software program EnSite, which is an embodiment of the present invention. Each step is explained in detail below.

1. The software requires as input the three dimensional (3D) coordinates of the enzyme, in the Protein Data Bank (PDB) format for this example, although of course other formats could optionally be used.

2. All the heteroatoms and hydrogen atoms listed in the PDB file are excluded.

3. The molecular surface [87, 88] can be computed using a variety of programs, such as the Accelrys package or the “MDS” subroutine (both are based on Connolly's publications [87, 88]), that produce dot representations of the surface. The executable of the MDS routine is available free on the web: www.biohedron.com/msp.html. Of course many programs may optionally be used as well, because items provided through stages 1-3 herein are input to the method of the present invention. Surface computation parameters: The Probe radius was set to 1.4; the surface density was set to 6.7 dots/Å²; the Accelrys-InsightII atomic van der Waals radii are used. The probe radius and surface dot density parameters are standard in computations of molecular surfaces with the Accelrys modeling package, which was used in this illustrative, non-limiting implementation of the present invention. Similarly the van der Waals atomic radii are standard values for the Accelrys-InsightII software. These parameters were also used with the MDS software as they are considered to be typical for the industry.

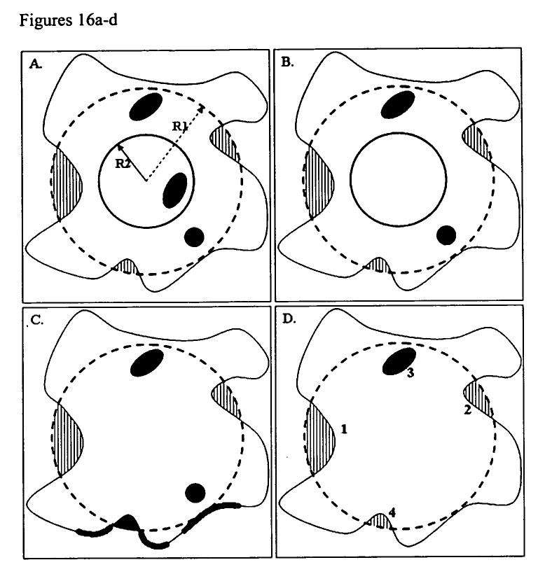

4. Computing the enzyme's centroid (Cmass) and selecting the surface dots within the 1^{st} sphere. EnSite computes the enzyme's centroid (Cmass) from the three-dimensional coordinates provided in step 1 above, and selects the surface dots within the 1^{st} sphere (the centroid is the average of the X, Y and Z coordinates of the atoms). The radius of the 1^{st} sphere, R1 (see FIG. 16) is chosen to include 5% of the surface dots which are nearest to the enzyme's centroid, but not more than 5% of Dsurface. The parameter Dsurface was set to 75000 surface dots, which is the average number of surface dots for the single domain monomers in dataset-I (see below). In practice R1 is the largest distance of any surface dot (R) within the 1^{st} sphere from the enzyme's centroid. Notably, the enzyme's centroid is used in the computational process for simplicity but Cmass could also be the center of mass of the enzyme. For enzymes with fewer surface dots than Dsurface, the radius of the 1^{st} sphere, R1 (see FIG. 16) is chosen to include 5% of the surface dots which are nearest to the enzyme's centroid. For larger enzymes, R1 is chosen to include a fixed number of surface dots (5% of Dsurface).

Dsurface—was set to 75000 surface dots which is the average number of surface dots for the single domain monomers in dataset-I. We assume that this average represents the average size of the basic enzyme structural unit. The typical size of a polypeptide chains is 30,000-50,000 Daltons [89]. A surface of 75000 dots corresponds to the lower end of this range, polypeptide chains of approximately 30,000 Daltons.

5. The first clustering step assembles the surface dots within the 1^{st} sphere into spatially distinct clusters. The grouping process starts from a yet ungrouped surface dot, which is farthest from Cmass, and looks for its neighbors within a specified Cdmax value (see below). The process is continued until all the neighboring surface dots are grouped and a cluster is defined. The clustering procedure is repeated for the remaining surface dots in the first sphere. This clustering stage is stopped when the distance from Cmass to the farthest ungrouped surface dot, is less than R2 (see FIG. 16). The R2 limit (R2<R1) helps to reduce the computation time, because the clusters formed within the R2 sphere are all internal voids. It is assumed that the chance of such clusters to be the active site decreases when they are deeper within the protein core.

R2—is set to be half of R1 and its purpose is to reduce the number of internal clusters within the 1^{st} sphere as described above.

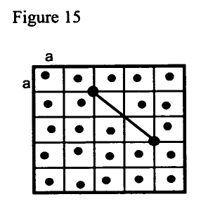

Cdmax—is the maximum distance to cluster two adjacent surface dots and it is set to 1 Å based on the density of surface dots used in this study, although of course this value could optionally be varied according to the density of surface dots used in the above calculations. The parameter was derived from the surface density of 6.7 dots/Å² which gives an average surface area for each surface dot of 0.149 Å². In this case each edge (represented as “a” in FIG. 15, which shows surface dot density. Circles represents surface dots. Squares represent the average surface area ascribed to each dot. The maximum theoretical distance between two adjacent surface dots is represented by the blue line and is equal to \sqrt{8α²}) is equal to 0.386 Å and Cdmax is equal to √8×0.386²=1.09 Å. The value was rounded off to 1 Å for ease of calculation and without intending to be limiting in any way. It should also be noted that all parameters given herein are intended as illustrative examples and are not meant to be limiting in any way; one of ordinary skill in the art could easily adjust these parameters.

6. Clusters which were identified in the previous step and which contain fewer surface dots than the adjustable parameter Mcluster are subjected to a second clustering stage in order to verify if they are small internal clusters (representing internal voids) or the beginning of a larger surface accessible cluster. All the other clusters are considered to be surface accessible. In the second clustering stage the center of mass of the cluster is computed and all the surface dots, which are outside the 1^{st} sphere yet located in the vicinity of the given cluster, are used as a reservoir in the second clustering process. The same parameters are used for increasing the clusters as in the first clustering stage. Therefore only surface accessible clusters or particularly large internal clusters will increase in this step. The second clustering process is stopped when the number of surface dots surpasses Mcluster. If the number of surface dots in the second clustering process for a given cluster is smaller than Mcluster, this cluster is discarded.

Mcluster—is the minimum number of surface dots in an internal cluster that renders it a putative active site cluster.

7. All the clusters are ranked according to the number of surface dots. For each enzyme only the first four clusters were considered in this study, hence the ranks are 1 to 4 or Not Found.

8. The output of the program is a ranked list of putative active sites. Each site is represented by the set of atoms that contribute to the surface formed by the cluster of surface dots that define the site.

FIG. 16 shows a schematic illustration of the main concepts in the active site prediction algorithm EnSite. A: The first clustering step: The blue circle represents the 1^{st} sphere, which includes 5% of the surface dots nearest to the enzyme's centroid. R1 is the radius of the 1^{st} sphere. The first clustering step can identify internal voids (represented by the small ellipsoids and circles) and surface accessible clusters (represented by the hashed blue areas). B: Clusters within a sphere of radius R2 are eliminated. C: A second clustering stage is applied only to small clusters with less than Mcluster surface dots (represented by the red areas). The thick blue lines represent the surface dots reservoirs for the second clustering process. The small internal void (red circular patch) fails to extend in the second clustering stage and it is eliminated. In contrast, the small open cluster near the bottom of the molecule can extend and it is considered a putative active site. D: ranking the final clusters according to their size.

Comparison of Predicted Active Sites to the Known Site

All 65 enzymes in dataset-I were checked manually, i.e. by visual inspection of the structure. The active site location was determined either by locating a bound ligand (most cases), and/or by searching the literature.

The catalytic residues in dataset-II are listed in the catalytic site atlas website and therefore the list of atoms for each predicted site was compared to the list of catalytic residues in the website. In cases in which more than one predicted site was a hit, the site with more predicted catalytic residues was considered. In case of evenness the higher rank was considered. Enzymes for which the catalytic site was not identified (24 enzymes) and 34 additional enzymes for which only one catalytic residue was identified (including those that have only one catalytic residue) were checked by visual inspection. Such inspection was necessary because in some cases the automatic procedure did not identify catalytic residues that were positioned near the active site rim; yet, it correctly locates the bottom of the active site. We included in the manual check predictions in which only one catalytic residue was identified because identification of a single point does not always provide the spatial location of the active site.

Structural Biological Molecule

The effect of the oligomerization state of the enzymes on the prediction performance was investigated. The structural biological molecule was taken either from the literature or PQS [90], that was kindly supplied by the generosity of Gail J. Bartlett.

Closeness Centrality Analysis

Closeness Centrality values were calculated using the SARIG server http://www.weizmann.ac.il/SARIG

Inaccessible Active Sites

Pocket areas were calculated using the castP server http://cast.engr.uic.edu/cast/

Approximate enzyme surface area was calculated by dividing the number of molecular surface dot (see the active site prediction algorithm section part 3) by the surface density 6.7 dots/Å².

Implementation to Docking

Docking solutions were computed by the MolFit software. Available at:

http://www.weizmann.ac.il/Chemical_Research_Support//molfit/

### Example 1

The catalytic residues are found in close proximity to the enzyme's centroid.

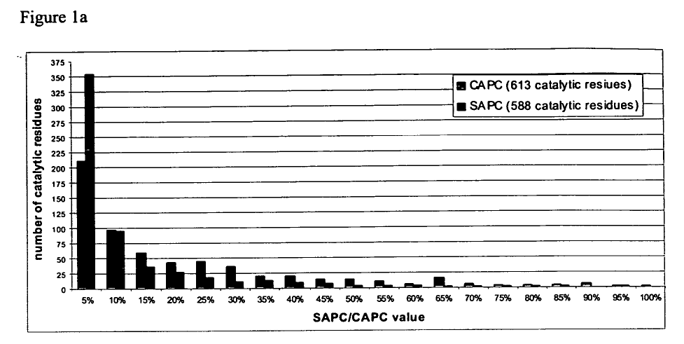

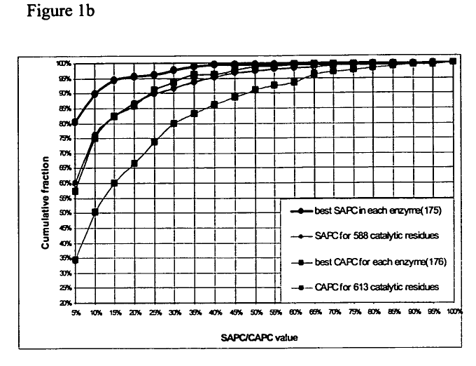

The concept that catalytic residues are positioned in close proximity to the molecular centroid was first validated in this set of experiments. This was done by calculating the distances of the catalytic residues in each enzyme in the dataset of Bartlett et al (176 enzymes) from the enzyme's centroid and comparing them to the corresponding distances for other residues. Two measures were employed, CAPC and SAPC (see Methods section). In the CAPC calculations all the 613 catalytic residues in the dataset were considered, whereas the SAPC analysis included only the 588 catalytic residues that contribute to the molecular surface. One PDB entry, 1 lnh, was excluded from the analysis because its single catalytic residue is found to have no surface accessibility. FIGS. 1a and 1b present the distribution of CAPC and SAPC values and the cumulative fraction of enzymes or catalytic residues with different CAPC and SAPC values.

FIG. 1a shows the distribution of catalytic residues according to their CAPC and SAPC values, which measure their distances from the molecular centroid relative to other residues (see Methods). Note the large proportion of catalytic residues which are in close proximity to the molecular centroid compared to the rest of the enzyme. This proportion increases markedly when only the surface residues are considered (SAPC values).

FIG. 1b shows analyses of the cumulative fractions of enzymes or catalytic residues with given SAPC or CAPC values (see Method for definition) for the dataset of Bartlett et al (dataset-II). Each enzyme is represented by the lowest SAPC or CAPC value for any of the catalytic residues.

Examination of FIG. 1 provides a very clear and distinct picture of the tendency of catalytic residues, and therefore of active sites, to be located in close proximity to the enzymes' centroids. This tendency is observed for the CAPC values, which compare all the Ca atoms in the enzyme, including those of the core residues, suggesting that this property is embedded in the enzyme structural scaffold. It is far more pronounced for the SAPC values which consider only exposed residues. Thus, for 80% of the enzymes in the dataset, at least one catalytic residue that contributes to the enzyme's surface is found among the 5% surface residues closest to the enzyme's centroid. The proportion increases to 94% when 15% of the surface residues closest to the centroid are considered (FIG. 1b). The SAPC values for the 175 enzymes are all in the range 0-35%, except for one outlier with SAPC=95%. A detailed examination of this enzyme (PDB code 1xva) reveals that it is a dimer in which each monomer is generally globular but it has an extended N-terminal tail that corks the entrance of the active site of the adjacent monomer. The only catalytic residue listed for this enzyme is found on the extended N-terminus [40]. Thus, when only one chain (monomer) is considered, the catalytic residue is found away from the monomer's centroid; yet the active site is situated very close to the other monomer's centroid. We conclude that catalytic residues tend to be located near the centroid of the enzyme. This new property is uniquely defined and easily measured and can therefore be used in different algorithms.

### Example 2

### Prediction of the Active Sites of Enzymes

The present Example includes experiments for determining whether the tendency of catalytic residues to be near the centroid of the enzyme can serve as a parameter for identifying active sites. This Example examines performance of a non-limiting exemplary implementation of the method according to the present invention, through use of the previously described software program EnSite, previously described in the Methods section, which tests only surface portions that are in close proximity to the centroid of the enzyme.

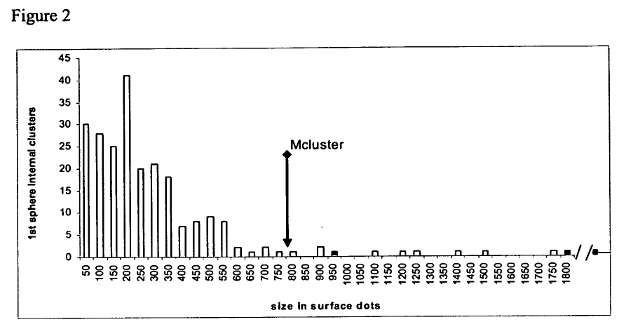

The main concept of EnSite is to look for clusters of surface dots within a small sphere around the enzyme's centroid. Since we look at the surface from the inside outwards there is no need to define a sea level from which the depth of the cavity is measured. Another result of the different point of view adopted here is that EnSite can also identify buried active sites. FIG. 2 shows the 1^{st} sphere internal clusters distribution in dataset-I. In three enzymes the internal cluster was found to be a buried active site (2dhc, 8acn, and 1gpb). These are marked as black columns.

To this end, we had to optimize the parameter Mcluster which is the minimum number of surface dots which differentiates internal voids from buried active sites. The distribution of internal clusters in the 1^{st} sphere (see Methods for definition) is presented in FIG. 2. At list one internal cluster was found for 58 out of the 65 enzymes in dataset-I (see Methods). 232 internal clusters were found with an average of 263 surface dots per cluster. Only the largest internal voids were included in the list of predicted active sites. A p-value of 0.05 was selected as the limit, corresponding to 750 surface dots (Mcluster). Only 12 internal clusters from 11 enzymes were large than Mcluster. 3 of them are buried active site (2DHC, 8ACN, 1GPB). In most of the other cases, 7 out of 9, the internal cluster is located next to the active site and it can be considered as part of it. It would be interesting to examine these internal voids in the context of enzyme functionality, but this is out of the scope of this study.

Application of EnSite to the Dataset of Laskowski et al (Dataset-I)

We first tested the performance of EnSite by applying it to 65 enzymes form dataset-I (see Methods for details). Although this dataset does not optimally represent all the known enzyme groups, it includes only monomeric enzymes and is therefore particularly useful for calibrating the different adjustable parameters in the algorithm. The main hypothesis which underlies our method is that the mass of the enzyme is “formed” around the functional site, which in turn is preferentially located near the center of mass. Hence it should be easier to predict active sites in monomeric enzymes because they are less likely to have more than one active site or other functional sites, such as recognition sites for other monomers and subunits. In addition, this dataset was used previously to test the performance of different active site prediction algorithms and therefore can serve for comparing the results.

TABLE 1
results for dataset-I

| | Number | Fraction | |
|---|---|---|---|
| | of | of | |
| Rank | enzymes | total | Cumulative |
| 1 | 58 | 89.2% | 89.2% |
| 2 | 3 | 4.6% | 93.8% |
| 3 | 1 | 1.5% | 95.4% |
| 4 | 1 | 1.5% | 96.9% |
| NF | 2 | 3.1% | |

EnSite correctly identified the active site for 63 of the 65 enzymes (97%) and ranked it 1-4. Moreover, in 58 cases the rank was 1 (89%) and in 61 cases it was 1 or 2 (94%), as demonstrated in Table 1. The distance of each cluster to the enzyme's centroid is defined as the shortest distance of any surface dot in the cluster to the centroid. Notably, although the predictions in EnSite are ranked according to the size of the surface dots cluster, in 90% of the cases in which the largest cluster (ranked 1) represented the correct active site, it is also closest to the enzyme's centroid. In 3 out of the 65 cases (1GBP, 2DHC, 8ACN) the active site is buried; it was nonetheless recognized, as an internal void, and in all three cases was ranked 1. For a detailed list of all 65 enzymes see Appendix 1.

In 2 out of the 65 cases in the dataset-I (enzymes 1PII and 1BLL) the active site was not identified by EnSite. Examination of the two structures revealed that each of these enzymes consists of 2 domains and in both cases the centroid of the enzyme is found near the interface of the two domains. The detailed discussion below substantiates the rationale behind our method, described with regard to FIG. 3, which shows whole protein analysis as compared to separate domain analysis for 1PII and 1BLL. The white arrows point at the centroid positions for the whole protein (A, C) or for each domain (B, D). The ligand is shown in purple and metal ions are shown in orange (CPK models). The yellow surface dots represent the largest cluster nearest to each centriod. A. 1PII—although the active site is partly recognized (near the phosphate ion in the left domain), most of it depicts the interface between the two domains B. 1PII—when the analysis considers each domain separately, the first cluster corresponds to the active site in each domain. C. For whole 1BLL the prediction failed as the first cluster is close to the interface of the two domains. D. For 1BLL-domain analysis, the 1st cluster identifies the bottom of the active site which is located in the large domain. Results for specific enzymes are described below.

Enzyme 1PII (EC-5.3.1.24 and E.C-4.1.1.48)—1PII is a monomeric bifunctional enzyme from E-coli, an N-(5′-phosphoribosyl) anthranilate isomerase and an indole-3-glycerol-phosphate synthase. It comprises two beta/alpha-barrel domains that superimpose with a root-mean-square (RMS) deviation of 2.0 Å for the common 138 Cα atoms. The C-terminal domain (residues 256 to 452) and the N-terminal domain (residues 1 to 255) catalyze two sequential steps in tryptophan biosynthesis. The two sites are identical as far as their positions within the tertiary structure of the beta/alpha-barrel is considered [41].

As aforesaid, one of the reasons for choosing the monomeric dataset was to eliminate the necessity to deal with more than one function. In this case there are two functions, each ascribed to a well defined structural domain. When both domains are treated as a single functional unit the calculated centroid is shifted to the interface of the two domains (FIG. 3a). In contrast, when each functional domain is treated separately both active sites are identified and ranked 1 (FIG. 3b).

This result supports our notion that enzymes evolved by distributing the mass around the active site. It is necessary to select the correct functional unit in order to obtain a good prediction of the active site. The proposition that each domain in 1PII should be considered as a separate functional unit is further supported by the observed deviation of the accessible surface area (ASA) of this protein form the empirical relation between ASA and the relative molecular mass. Such a correlation was presented by Miller et al (ASA=6.3 Mr^{0.73}±4%, where Mr is the relative mass) [42], who also pointed out that the correlation is valid for monomeric multi-domain proteins and single-domain proteins. A similar relation for globular oligomeric proteins was established by Janin et al [43]. The ASA of the enzyme 1PII is 9.4% larger than the expected value for a monomeric protein of the same molecular mass [41], whereas the ASA values for the separate domains are only 1% and 0.3% smaller than the expected values. Morever, indole-3-glycerol-phosphate synthase exists as a separate enzyme in most microorganisms [44]. In the 1PII enzyme, evolution selected to join together the two sequential functions in the same biosynthesis pathway.

Enzyme 1BLL (EC-3.4.11.1)—this is the bovine lens leucine aminopeptidase (bILAP) [45]. Its two domains are 162 and 318 amino acids long and the larger domain contains the active site. Like in the 1PII case, when analyzing both domains as the functional unit, the centroid is positioned at the interface of the two domains and the active site is not identified (FIG. 3C). When each domain is treated separately the algorithm correctly identifies the active site in the larger domain. The prediction is ranked 1 and includes all three catalytic residues (Asp-255, Arg-336 and Lys-262) (FIG. 3D).

Application of EnSite to the Dataset of Bartlett et al (Dataset-II)

The recently published dataset of Bartlett et al [18, 46] includes 178 enzymes, of which 176 were included in our study (see Methods). Prediction results for this dataset are summarized in Table 2.

TABLE 2
results for dataset-II

| | Number | Fraction of | |
|---|---|---|---|
| Rank | of enzymes | total | Cumulative |
| 1 | 130 | 73.9% | 73.9% |
| 2 | 14 | 8.0% | 81.8% |
| 3 | 5 | 2.8% | 84.7% |
| 4 | 3 | 1.7% | 86.4% |
| NF | 24 | 13.6% | |

EnSite identified the active site and ranked the prediction 1-4 in 152 cases (86%). In 130 cases (74%) the rank of the correct prediction was 1 and in only 3 cases it was 4. Also, for 120 of the 130 cases in which the prediction was ranked 1 (92%) the largest cluster was closest to the enzyme centroid. Notably, the best results were obtained for moderate size enzymes. Thus, for enzymes with surface area between 5500 Å² to 20500 Å² (80% of the data) EnSite ranked the correct prediction 1 in 78% of the cases and 1-4 in 91% of the cases. For 5 enzymes the active site is buried. EnSite identified it in all 5 cases as an internal cluster and ranked it 1 or 2. A detailed list of the results for all 176 enzymes is given in appendix 2.

Dataset-II shows the effect of the oligomerization state of an enzyme on the prediction performance of EnSite. Dataset-II includes multimeric enzymes, thereby allowing us to test the effectiveness of EnSite in cases in which the active biological unit is an oligomer. In this test, the 176 enzymes were divided into 3 groups: monomers, homo-multimers, and hetero-multimers. Notably, the monomers comprise only 30.7% of this dataset, which contains 59.1% homo-multimers and 10.2% hetero-multimers. Correct predictions for 81% of the monomers were ranked 1 with overall successes rate (rank 14) of 89%. Slightly lower values were obtained for homo-multimers: 76% correct predictions were ranked 1 and the overall success rate was 87.5%. The prediction success rate is however, lower for hetero-multimers—in 55% of the cases a correct prediction was ranked 1 and the overall success rate was 72%.

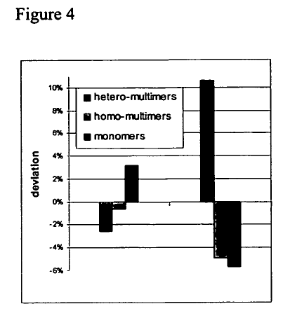

FIG. 4 shows the deviation of the percent of correct predictions ranked 1 or NF in different biological structural groups (monomers, homo-multimers, hetro-multimers) from the distribution of enzymes with different biological units in dataset-II.

Table 3 compares, for each structural group, the fraction of predictions ranked 1 or “Not Found” (NF) to the fraction of the enzymes in the different groups in the dataset. FIG. 4 shows the differences (in percent) between these fractions. It appears that the group of enzymes for which the correct prediction is ranked 1 is enriched in monomeric enzymes (33.8% predictions ranked 1 versus 30.7% monomers in the whole dataset) and depleted of hetero-multimeric enzymes (7.7% versus 10.2%). The opposite trend is observed for the group of enzymes for which the active site was not identified by EnSite. Thus, the prediction success of EnSite is anti-correlated to the biological complexity of the enzymes; it is most successful for monomeric enzymes and least successful for multimeric enzymes which comprise different polypeptide chains.

TABLE 3

| | | | | | Number of | Fraction of |
|---|---|---|---|---|---|---|
| | | Fraction in | Number of | Fraction of | enzymes | predictions |
| | Number of | dataset-II | enzymes | predictions | ranked | ranked |
| | enzymes | (176) | ranked 1 | ranked 1 | NF | NF |
| Hetero- | 18 | 10.2% | 10 | 7.7% | 5 | 20.8% |
| multimers | | | | | | |
| Homo- | 104 | 59.1% | 76 | 58.5% | 13 | 54.2% |
| multimers | | | | | | |
| Monomers | 54 | 30.7% | 44 | 33.8% | 6 | 25.0% |

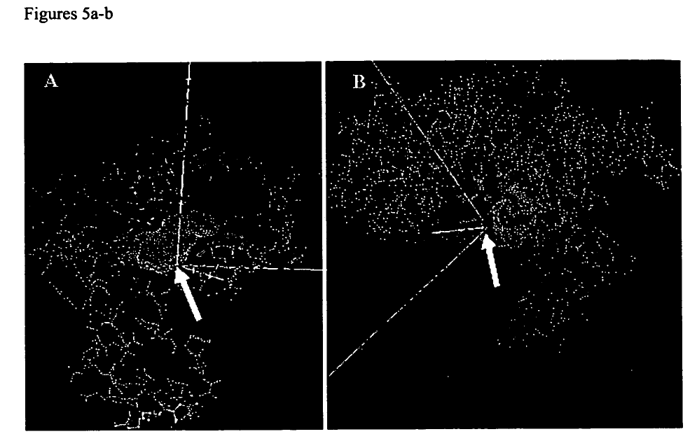

Only 4 enzymes out of 176 in dataset-II are heteromers in which the catalytic residues are located on more than one chain. In 3 cases they are found on two chains (pdb codes: 1AHJ, 1BCR, 1MHL) and in one case the catalytic residues are found on 3 different chains (1PYA). EnSite was applied to each chain separately and to all the chains in the enzyme together. In the latter approach, i.e. when the chains that carry catalytic residues were treated as a single structural unit, the algorithm was able to locate the active site in all 4 cases, and it ranked the correct prediction as 1 (see table 5). Analysis of each chain separately also identified the relevant catalytic residues (accept in 1PYA) but the recognition usually improved when the chains were combined (see table 4). Two examples are presented in FIG. 5.

Table 4 shows comparison of the prediction for separated chains and combined chains in 4 heteromers. The catalytic residues are located in more than one chain.

TABLE 4

| | |
|---|---|
| separated chains | combined chains |

| | recognized | recognized | recognized | recognized |
|---|---|---|---|---|
| | catalytic | catalytic | catalytic | catalytic |
| | residues | atoms | residues | atoms |
| 1MHL | 2 | 11 | 3 | 14 |
| 1BCR | 5 | 17 | 4 | 11 |
| 1AHJ | 3 | 14 | 4 | 16 |
| 1PYA | 2 | 3 | 4 | 13 |

Separated chains—the algorithm was applied to each chain separately and the cumulative number of recognized catalytic residues/atoms from both chains is presented.

Combined—all the chains were treated as a single structural unit.

TABLE 5

| | | | | Number of | Number of | |
|---|---|---|---|---|---|---|
| | | | Number of | recognized | recognized | |
| E.C | | | catalytic | catalytic | catalytic | |
| number | PDB code | chain | residues | residues | atoms | Rank |
| 1.11.1.7 | 1MHL | C | 1 | 1 | 8 | 1 |
| | | A | 2 | 1 | 3 | 1 |
| | | A, C | 3 | 3 | 14 | 1 |
| 3.4.16.6 | 1BCR | A | 3 | 3 | 8 | 1 |
| | | B | 2 | 2 | 9 | 1 |
| | | A, B | 5 | 4 | 11 | 1 |
| 4.2.1.84 | 1AHJ | B | 1 | 1 | 9 | 1 |
| | | A | 3 | 2 | 5 | 1 |
| | | A, B | 4 | 4 | 16 | 1 |
| 4.1.1.22 | 1PYA | B | 2 | 2 | 3 | 3 |
| | | A | 1 | 0 | 0 | n.f |
| | | C | 1 | 0 | 0 | n.f |
| | | A, B, C | 4 | 4 | 13 | 1 |

Table 5 shows separated and combined chain analysis for 4 heteromers in which the catalytic residues are found in more than one chain.

FIG. 5 shows examples of enzymes in which the catalytic residues reside in two different chains. Different blue shades represent different chains. The catalytic residues are colored in green; the white arrow point at the centroid location and the yellow dots represent predicted active site. A. 1BCR with 5 catalytic residues (A53, A146, A147, B338, B397). B. 1AHJ with 4 catalytic residues (A113-A115, B56).

The effect of multi-domain structures on the prediction performance was then determined. As described with regard to Table 2, our program correctly identified the enzyme's active site and ranked it 1-3 in 149 out of the 176 cases. Next, we checked in detail the 27 cases for which the program did not identify the active site (24 NF cases) or ranked it 4 (3 cases). the CATH database provides domain classification for 25 of the 27 cases [47, 48]. For 13 of them the enzyme consists of more than one domain. This allowed a more extensive analysis of the effect of multi-domain structures on the prediction ability (a limited analysis is described above for 2 enzymes from dataset-I). All the domains which include at least one catalytic residue were included in this analysis. When catalytic residues were found to be located in more than one domain, additional analysis considered these domains as a single functional unit (“combined domains” analysis). One exception is the enzyme 1QPR, in which two domains from different chains were combined (FIG. 6b). In total, 13 multi-domain enzymes were analyzed and the results are summarized in Table 6, which shows domain analysis of 13 multi-domain enzymes. The analysis was performed on domains which include the catalytic residues. For enzymes in which the catalytic residues located in more than one domain, additional analysis was performed (combined domains), considering the domains as one structural unit.

TABLE 6

| | | | Rank | | | | | | |
|---|---|---|---|---|---|---|---|---|---|
| | | | for | Number | | Number of | Number of | Number of | |
| | E.C | PDB | single | of | Domain name | catalytic | recognized | recognized | |
| | number | code | chain | domains | (by CATH) | residues | catalytic residues | catalytic atoms | Rank |
| 1 | 2.1.1.72 | 2ADM | NF | 2 | 2ADMA1 | 3 | 3 | 22 | 1 |
| 2 | 6.3.5.2 | 1GPM | NF | 3 | 1GPMA1 | 5 | 4 | 13 | 1 |
| 3 | 3.5.1.5 | 1KRB | NF | 2 | 1KRBC2 | 4 | 2 | 3 | 1 |
| 4 | 5.99.1.2 | 1I7D | NF | 4 | 1I7DA1 | 2 | 2 | 7 | 2 |
| | | | | | 1I7DA4 | 2 | 1 | 3 | NF |
| | | | | | A1 + A4 | 4 | 4 | 24 | 1 |
| 5 | 1.14.13.7 | 1FOH | NF | 3 | 1FOHA1 | 1 | 0 | 0 | NF |
| | | | | | 1FOHA3 | 2 | 1 | 3 | 1 |
| | | | | | A1 + A3 | 3 | 2 | 4 | 1 |
| 6 | 6.5.1.2 | 1DGS | NF | 6 | 1DGSA2 | 3 | 1 | 4 | 1 |
| | | | | | 1DGSA3 | 1 | 1 | 1 | 1 |
| | | | | | A2 + A3 | 4 | 3 | 13 | 1 |
| 7 | 3.1.3.2 | 4KBP | NF | 2 | 4KBPA2 | 3 | 2 | 2 | 4 |
| 8 | 5.3.1.25 | 1FUI | NF | 3 | 1FUIA2 | 1 | 1 | 1 | 2 |
| | | | | | 1FUIA3 | 1 | 1 | 2 | 2 |
| | | | | | A2 + A3 | 2 | 2 | 5 | 4 |
| 9 | 1.1.3.9 | 1GOG | NF | 3 | 1GOG02 | 4 | 1 | 3 | NF |
| 10 | 4.3.2.2 | 1C3C | NF | 3 | 1C3CA2 | 2 | 0 | 0 | NF |
| 11 | 1.7.2.1 | 1NID | NF | 2 | 1NID01 | 1 | 0 | 0 | NF |
| | | | | | 1NID02 | 1 | 0 | 0 | NF |
| | | | | | 01 + 02 | 2 | 0 | 0 | NF |
| 12 | 2.7.7.12 | 1HXQ | 4 | 2 | 1HXQA2 | 4 | 2 | 8 | 1 |
| 13 | 2.4.2.19 | 1QPR | 4 | 2 | 1QPRA1/B1 | 1 | 1 | 5 | 1 |
| | | | | | 1QPRA2/B2 | 3 | 2 | 4 | 1 |
| | | | | | A1 + B2 | 4 | 4 | 16 | 1 |

When the functional unit of the enzyme (the domain or domains which include the catalytic residues) is considered in the prediction the algorithm was able to identify successfully the active site in 10 of the 13 cases (77%) mentioned above, ranking it 1 in 8 cases and 4 in the 2 other cases. Among the 13 multi domain enzymes, 6 were found to have catalytic residues in more than one domain. For 5 of them analysis of “combined domains” improved the active site prediction. Notably, in 4 of these 5 cases analysis of each domain separately gave good results, yet the combination of the functional domains consistently improved the results, increasing the number of recognized catalytic residues and catalytic atoms (see FIGS. 6, 7). In one case (1nid) a correct predictions was not obtained for either the separate domain or the combined domains.

FIG. 6 shows separate and combined domain analysis in cases in which the catalytic residues (depicted in green) are located on more than one domain. The ligands are colored white. The arrows point at the centroid of the combined domains. A. 1FOH, Phenol Hydroxylase complex with FAD (ball and stick) and phenol (CPK). The active site is formed by the two upper domains (blue and purple). The red dots represent the predicted active site when each domain was considered separately. The yellow dots represent the predicted active site when the two domains were considered as combined structural unit. B.1QPR, Quinolinate Phosphoribosyltransferase complex with phathalic acid (CPK). The two chains are colored in different blue shades. Two identical active sites are formed by chain dimerization; each site is located on two domains. The predicted active site for the combined domain is shown in the upper part of the figure. The predictions for the separated domains are shown in the bottom part of the figure.

FIG. 7 shows catalytic atoms recognition in domain analysis, in cases in which the catalytic residues are located in two different domains. Separated domains—the algorithm was applied to each domain separately and the cumulative number of recognized atoms in both domains is presented. Combined—both domains were considered as a single structural unit.

Next, 13 enzymes were examined for which Ensite was unable to predict the correct active site, yet which are not multi-domain enzymes.

As mentioned above, EnSite failed to identify the correct active site for 24 of the 176 enzymes. Eleven of them are multi-domain enzymes. The results for the other 13 are analyzed in this section. For two monomeric enzymes in this group the rank of the correct active site was larger than 4 and therefore considered as NF.

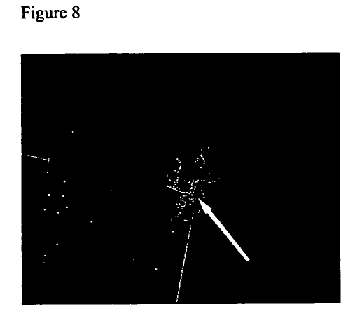

The first case is 2ACY, an acylphosphatase which is one of the smallest enzymes known (˜10 KDa) [49]. This enzyme and the effect of enzyme size on the prediction performance are discussed in the algorithm limitations subsection. The second case is 1CHD, the C-terminal (residues 152-349) catalytic domain of chemotaxis receptor methylesterase [50]. It appears that the catalytic activity of this enzyme is not confined to the C-terminal domain. Enzyme kinetics studies showed that the two domains phosphorylated protein has significantly higher methyl-esterase activity than the isolated C-terminal domain [51]. Application of EnSite on both domains as a single structural unit (pdb 1A2O) resulted in a correct active site prediction (at the interface of the two domains) ranking 1. FIG. 8 shows a model of the chemotaxis receptor methylesterase (1A2O). The C-terminal catalytic domain (1CHD) is shown by the sticks diagram and the N-terminal regulatory domain and the linker (bottom right) are shown in CPK diagram. The catalytic residues are colored in red. The white arrow points at the centroid location. The yellow dots represent predicted active site.

Ten of the 13 cases are multimers in which the active site is formed by more than one chain. These multimeric enzymes, are usually complex biological structures i.e. they include a large number of chains that form complicated morphological shapes like cylinders. They are also characterized by large interfaces between the chains and many active sites per enzyme. Only one structure in this group is a homo dimer although homo dimers comprise approximately 50% of the multimeric structures. The rest of the structures are: 2 trimers, 2 tetramers, 2 hexamers, an octamer, a decamer and a 24mer. This group was reanalyzed by applying the algorithm to the chains that form the active site combined together. Two parameters were modified: the Mcluster was set to 1500 to eliminate most of the internal voids which are found between chains; Dsurface was set to be a large number in order to fit the relatively big structures which are formed by combining the chains (see methods).

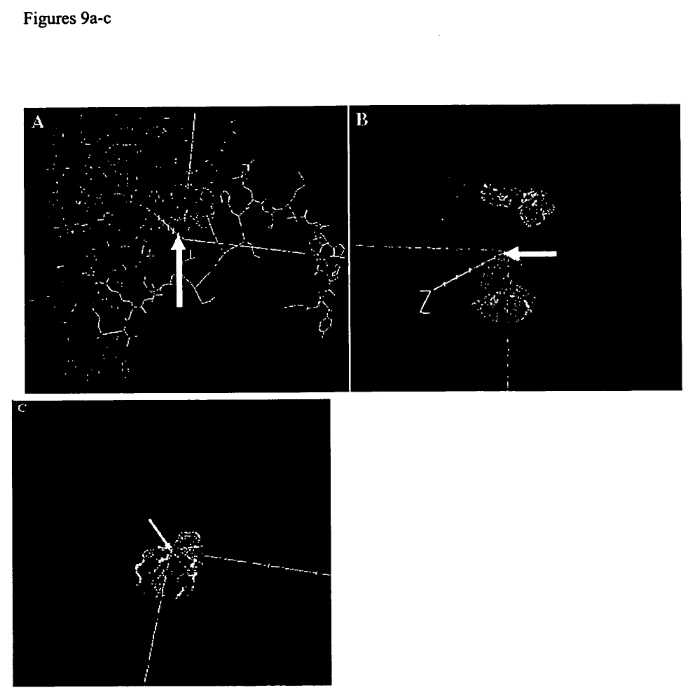

Examples of active sites formed by more than one chain although the catalytic residues (shown in green) are located on a single chain are shown in FIG. 9. The different chains are presented by different colors. The ligand bound in the active site is shown in red. The white arrow points at centroid location and the yellow dots represent the predicted active site. A. 1AW8: the active site (with a single catalytic residue) is formed by 4 chains. The predicted sites ranked 2 and 3 are presented. B. 1D4A: a homo-dimer. Two similar active sites are formed, each containing three catalytic residues and a bound FAD molecule. The predicted sites ranked 1 and 3 are presented. The site ranked 2 is a large internal void located inbetween the two catalytic sites (not shown). C. 1LXA: a homo-trimer in which three similar active sites are formed between adjacent chains.

In 6 cases (1AW8, 1DW9, 1D4A, 1FUG, 3PCA, 1LXA) the procedure helped to identify the correct site. FIG. 9a presents the case of 1AW8, in which the active site is formed by 4 chains. In the two structures 1D4A (FIG. 9b) and 1FUG, two identical active sites are formed; in both cases both active sites are predicted correctly. In 4 additional cases combining the chains was not helpful. Interestingly, 3 of the cases have 3-fold symmetry (2 hexamers and 1 trimer). Hence 1LXA is the only trimer in which the combined chain analysis was helpful (FIG. 9c). Finally in enzyme 1KFU, which is the structure of the full-length heterodimeric [L chain 80-kDa+S chain 30-kDa] human m-calpain (calcium-dependent cytoplasmic cysteine proteinases) [52], the L chain (699 residues) which includes the catalytic domain (residues 2-356) [52, 53] is a multi domain structure. Applying the algorithm to the catalytic domain results in a correct prediction ranked 1. It was not included among the multi-domain structures because it is not defined as such in the CATH database.

### Example 3

### Comparison to Other Geometry Based Active Site Prediction Algorithms

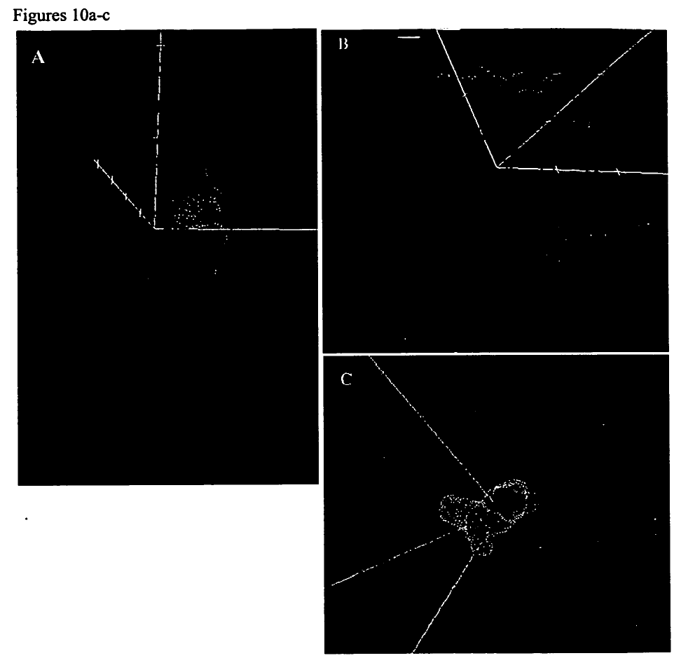

Laskowski et al, which constructed the monomeric dataset, used the SURFNET program [30] and showed that for 83% of the investigated enzymes the largest cleft, as defined by SURFNET corresponded to the actual active site [23]. EnSite ranked a correct prediction at the top for 89% of the enzymes in this dataset. 5 enzymes were classified by Laskowski et al as class 3, meaning that the correct active site was ranked 3 or more or it was not found. For All 5 cases EnSite correctly predicted the active site and rank it 1. Interestingly, these 5 enzymes have small, flat or buried active sites, emphasizing the advantage of our method for locating non trivial active sites. Thus, in enzyme 1ADD the ligand fits into the bottom of a deep cleft, yet the binding site is too wide to be detected by SURFNET (see FIG. 10c); in 8ACN the active site is buried, and SURFNET could not find it; in 1HNE the active site is small and shallow and therefore the correct prediction was ranked 6; in 2DHC the correct prediction is ranked 15 because the active site is small (EnSite identifies it as an internal void); and in 1RNH the active site is too flat to be detected by SURFNET.

In the latter case conservation and mutation analysis proposed 3 catalytic residues [54, 55] all of which are recognized by our algorithm (see FIG. 10a).

Liang et al analyzed the same dataset using an early version of the CAST program, which is based on the alpha shape and the discrete flow theories [25]. They omitted 14 enzymes from the dataset because for their active/binding sites the discrete flow went to infinity. The authors used the analogy of a “soup plate” to describe the geometrical shape of these 14 active sites. For the other enzymes the prediction rate was moderate. Thus, even after eliminating 14 enzymes, the best results were obtained for a group of 39 enzymes in which 74% were characterized as rank 1. It appears that although the CAST method locates, measures and defines surface pockets well, it has difficulty in discriminating the correct pocket from other surface pockets.

Examples of shallow/flat active sites are shown in FIG. 10. The ligand bound in the active site is shown in CPK. The yellow dots represent the predicted active site. A. Ribonuclease H (1RNH). The three catalytic residues are shown in red ball & stick. B. The achromobacter protease. The prediction was made for the apoprotein 1ARB. The position of the ligand was taken from the structure of the bound enzyme 1ARC. C. Adenosine deaminase (1ADD) with bound 1-deazaadenosine.

Peters et al using the APROPOS program tested 24 different structures of enzymes from the subtilisin protease family and reported good results. Hence, their program failed to recognize the active site in only one case—the achromobacter protease (1ARB), which has a very flat active site [27]. This unique flat active site of the achromobacter protease was also observed by Phillips et al who investigated 62 different proteases [56]. EnSite recognizes the achromobacter protease active site and ranked the correct prediction 1 (see FIG. 10b) thus, although the site is very flat, it contains the largest surface portion in the 1^{st} sphere.

Inaccessible active sites are also problematic for many prediction programs. Bartlett et al found that 5% of all the catalytic residues have 0% relative surface area (RSA) [18]. Indeed in a few enzymes the active site appears to be totally inaccessible. Most of the existing geometry based algorithms have difficulty to locate such active sites. An example is the active site in 8ACN for which the SURFNET program failed to locate the active site as discussed above. Even algorithms which are designed to deal with internal voids (like the CAST algorithm) often have difficulties in the ranking of such a predictions because of the differences in size compared to regular external cavities which are usually much bigger. The positive correlation between the number of pockets/cavities and protein size [25], further contributes to the difficulty in ranking buried active sites, as the total number of false sites increases with protein size.

Table 7 shows a comparison between the performance of EnSite and CAST for 5 enzymes with buried active sites.

TABLE 7

| | | Enzyme | | | | |
|---|---|---|---|---|---|---|
| | | surface | | | CAST | CAST |
| | PDB | area | EnSite | CAST | Number | Pocket |
| E.C Number | code | [Å²] | rank | rank | of pockets | area |
| 2.4.1.1 | 1GPA | 35423 | 1 | 1 | 153 | 1882.8 |
| 2.3.1.54 | 2PFL | 27681 | 2 | 14 | 127 | 122 |
| 1.14.13.25 | 1MHY | 22300 | 2 | 8 | 88 | 188.3 |
| 3.8.1.2 | 1QQ5 | 10124 | 2 | 2 | 30 | 141.1 |
| 4.2.1.3 | 1FGH | 26885 | 1 | 5 | 100 | 366.4 |

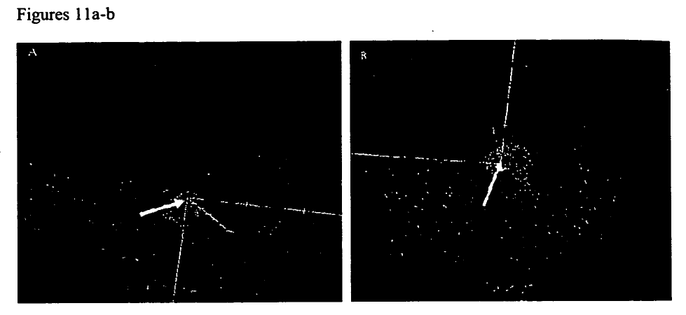

Buried active sites were found in 5 enzymes in dataset-II (for examples see FIG. 11, which shows examples of buried active sites. The catalytic residues are colored in red. The white arrow points at the centroid location and the yellow dots represent the predicted active site. A. 1QQ5, B. 1FGH.) and all were identified by EnSite, and ranked 1 or 2. We also analyzed these 5 cases with the CAST program using the castP server [25, 33]. The CAST program defined these internal pockets better than our algorithm, namely in some cases EnSite found most of the internal pocket but not all of it. Yet, the ranking by EnSite was always better or equal to the ranking by CAST (see Table 7) as all 5 enzymes were ranked 2 or lower with CAST.

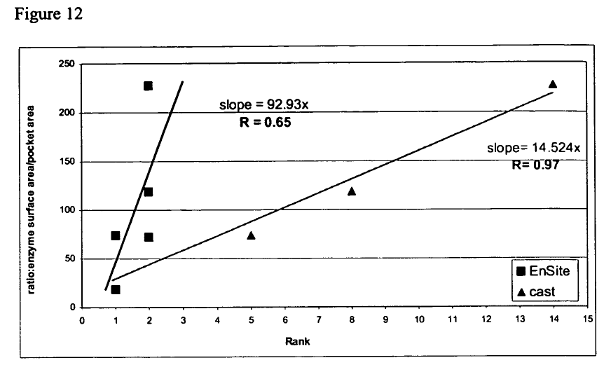

Next, we checked if the ranking of buried active sites is affected by the enzyme's size and/or by the internal pocket size. A strong correlation is observed between the CAST ranking of internal active sites and the ratio between the surface size to active site size (R=0.97, see FIG. 12 which shows the dependence of the rank of the correctly predicted site and the ratio of enzyme surface area to pocket area for 5 enzymes with buried active sites), hence, for a larger protein and/or smaller internal pocket the rank is likely to be lower. The EnSite ranking shows a similar general tendency but as the ranks are 1 or 2, the dependence on this ratio is barely significant. Hence, the EnSite ranking is clearly less sensitive to the relative size of the buried active site and the enzyme size.

Recently the connectivity within enzymes was analyzed using the dataset of Bartlett et al (dataset-II). Proteins were transformed into mathematical graphs (networks) [39], in which the amino acids are the nodes and their interactions are the edges. It was found that the closeness centrality values for active site residues are higher than those of other residues in the enzyme. Namely, active site residues interact with most other residues, either directly or by few intermediates. By combining closeness and RSA values for each amino acid, correct partial predictions of the active site were obtained for 70% of the investigated enzymes.

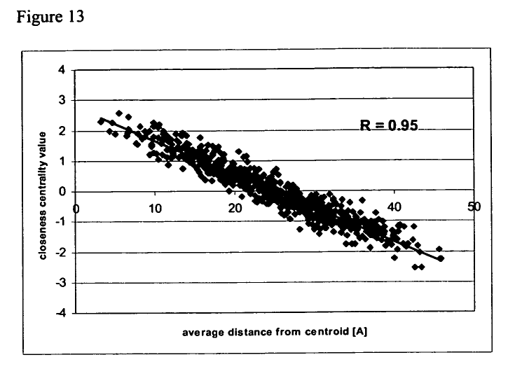

We examined the relation between the amino acid closeness values and their average distance from the protein centroid for 5 randomly selected structures (1AB8, 1CHD, 1G72, 1WG1, 1FOH). The closeness values were calculated using the SARIG server (http://www.weizmann.ac.il/SARIG). The correlations coefficients are in the range 0.94-0.96 (see example in FIG. 13, which shows the correlation between the closeness centrality values computed with the SARIG server and the average distance from enzyme centroid for the 662 amino acids of 1FOH) indicating that the centrality of the active site residues is achieved by placing them near the enzyme's centroid.

### Example 4

### Application to Enzyme Inhibitor Docking

In the most general form docking procedures use the coordinates of the unbound component molecules and no additional data is provided. However, docking of unbound structures is difficult and in practice, biochemical, biophysical or other data are often used in order to improve the docking results [57, 58]. Thus, the sites recognized by EnSite can be used in order to upweight the relevant portion of the surface in docking searches. However, finding a new source of data is more beneficial, especially if these data are not sensitive to conformational changes.

The docking algorithm (MolFit) which was developed in our group, identifies and quantifies surface complementarity [59-61]. In this algorithm and in many others, the role of the geometric complementarity term is dominant [62]. The fact that the enzyme's activity is inhibited in most cases by active site blocking, together with our finding that active sites are positioned in close proximity to the enzyme centriod led us to test a new filter of docking solutions, namely re-ranking by the distance of the interface to the enzyme centroid.

TABLE 8

| | | MolFit | | Rank | |
|---|---|---|---|---|---|
| | System | geometric rank | Rms | by distance | Rms |
| 1 | 1tec | 184 | 6.47 | 7 | 3.52 |
| 2 | tem1/blip | 1 | 3.55 | 2 | 3.55 |
| 3 | 2sec | 947 | 2.86 | 2 | 2.86 |
| 4 | 1avw | 2343 | 6.8 | 1 | 6.8 |
| 5 | 1fss | 699 | 2.37 | 97 | 7.01 |
| 6 | 1cho | 136 | 1.8 | 2 | 1.8 |
| 7 | 2ptc | 278 | 5.93 | 24 | 6.68 |
| 8 | 1bth | 519 | 9 | 41 | 8.6 |
| 9 | 1brs | 10 | 4.49 | 30 | 5.74 |

To this end we selected 9 enzyme inhibitor complexes for which geometrical docking was previously preformed [63, 64]. Each run produced 8760 solutions ranked by their geometric score. For each solution the distance from the enzyme's centroid was calculated for each atom in the inhibitor. Next, the 10 closest atoms were identified, and the distance between their centroid and the enzyme's centroid was used to re-rank the solutions. A nearly correct solution was identified as follows: for each prediction the Cα atoms of the enzyme were superposed on the enzyme in the experimental structure of the complex, and then the RMSD between the common Cα atoms of the inhibitors in the two structures (ligand RMSD) was calculated. Next, we searched the sorted list for the highest ranking solution with ligand RMSD lower then 8 Å (one exception is the system 1bth for which an RMSD limit of 9 Å was used because of the large conformation change that accompanies the complex formation). Table 8 compares the rank of the nearly correct solution according to the geometrical score and according to the distance of the enzyme inhibitor interface to the enzyme centroid in unbound docking. The RMSD values were computed for the ligand after optimal superposition of the enzyme. The results are strikingly better in almost every case (1brs being the exception). The re-ranking by distance from the enzyme's centroid improved the ranking of the nearly correct solution dramatically. The most striking result was achieved for 1avw for which the rank improved from 2343 to 1. The fact that no preliminary data on the enzyme active site is needed makes this method very simple and useful.

If a correct solution is not found in the list of solutions produced by the docking program it will not be identified in the re-ranking process. It is possible however to use the predictions of EnSite to upweight the relevant portions of the surface in the docking scan (thereby improving the chance that the correct solution is in the list of solutions) and than to use the distance filter.

### Example 5

### Problems and Future Directions

The ultimate goal of active site prediction algorithms is to locate the correct active site and rank it high. As was shown by the comparison to the CAST program (see FIG. 12, table 7), methods which define all possible cavities on the enzyme surface, have difficulty in pointing out the correct active site among the large number of false sites. The limited search performed by our algorithm reduces significantly the number of false predictions.

The close proximity property on which EnSite is based was shown to be dependent on the functional unit of the enzyme. Thus, except for one structure (2ACY a very small monomer; see below) all the NF cases are complex multi-chain enzymes, in which the active site is formed by more than one chain or they are multi-domain enzymes. We assumed that if the functional unit is correctly defined, than the active site will be included among the top predicted surface portions. Therefore, in contrast to other methods EnSite is currently limited to consider only the 4 largest surface portions. Consequently, when a correct prediction is produced by EnSite, it is also ranked high. However, this limitation is also the cause of the main disadvantage of the current version of EnSite—the relatively high number of NF cases compared to other methods.

A potential non-limiting way to improve our method is to develop an algorithm which is able to identify the functional unit of the enzyme. With the current version of EnSite the problem can be circumvented by using the available biological knowledge in order to define better the functional unit.

EnSite best results were obtained for moderate size enzymes (80% of the data). The lower prediction success for large enzymes is attributed to the large fraction of multi-domains structures among them. As for small enzymes (5 structures), 4 of them are short polypeptide chains in which the functional unit includes more than one chain whereas only one is a functional monomer. This monomer is the structure of acylphosphtase (2ACY), which is one of the smallest enzymes known (˜10 KDa) [49]. EnSite identified 11 surface portions within the 1^{st} sphere for this enzyme, which is the highest number of predicted surface portions in the entire dataset and more than three times of the average, indicating that no dominant surface portion can be found. It is possible that the proximity property is less pronounced in very small enzymes.

Based on the current results a more sophisticated algorithm may be optionally developed that tests several functional units of the enzyme, mainly based on the biological unit and the domain classification. Active sites can be predicted for each putative functional unit but the ranking will consider all the predictions together. Formation of continuous sites (see FIG. 7) may be used as a measure for preferring one functional unit over another. In addition, the EnSite algorithm can be combined with one of the existing methods which “looks at the protein from the outside”, in order to maximize the extent of prediction success.

### REFERENCES

1. Abagyan, R. and M. Totrov, High-throughput docking for lead generation. Curr Opin Chem Biol, 2001. 5(4): p. 375-82.
2. Maggio, E. T. and K. Ramnarayan, Recent developments in computational proteomics. Drug Discov Today, 2001. 6(19): p. 996-1004.
3. Bohacek, R. S., C. McMartin, and W. C. Guida, The art and practice of structure-based drug design: a molecular modeling perspective. Med Res Rev, 1996. 16(1): p. 3-50.
4. Hubbard, R. E., Can drugs be designed? Curr Opin Biotechnol, 1997. 8(6): p. 696-700.
5. Klebe, G., Recent developments in structure-based drug design. J Mol Med, 2000. 78(5): p. 269-81.
6. Gane, P. J. and P. M. Dean, Recent advances in structure-based rational drug design. Curr Opin Struct Biol, 2000. 10(4): p. 401-4.
7. Burley, S. K., et al., Structural genomics: beyond the human genome project. Nat Genet, 1999. 23(2): p. 151-7.
8. Armon, A., D. Graur, and N. Ben-Tal, ConSurf: an algorithmic tool for the identification of functional regions in proteins by surface mapping of phylogenetic information. J Mol Biol, 2001. 307(1): p. 447-63.
9. Aloy, P., et al., Automated structure-based prediction of functional sites in proteins: applications to assessing the validity of inheriting protein function from homology in genome annotation and to protein docking. J Mol Biol, 2001. 311(2): p. 395-408.
10. Lichtarge, O., H. R. Bourne, and F. E. Cohen, An evolutionary trace method defines binding surfaces common to protein families. J Mol Biol, 1996. 257(2): p. 342-58.
11. Landgraf, R., I. Xenarios, and D. Eisenberg, Three-dimensional cluster analysis identifies interfaces and functional residue clusters in proteins. J Mol Biol, 2001. 307(5): p. 1487-502.
12. Dietmann, S. and L. Holm, Identification of homology in protein structure classification. Nat Struct Biol, 2001. 8(11): p. 953-7.
13. Orengo, C. A., D. T. Jones, and J. M. Thornton, Protein superfamilies and domain superfolds. Nature, 1994. 372(6507): p. 631-4.
14. Stark, A., S. Sunyaev, and R. B. Russell, A model for statistical significance of local similarities in structure. J Mol Biol, 2003. 326(5): p. 1307-16.
15. Wangikar, P. P., et al., Functional sites in protein families uncovered via an objective and automated graph theoretic approach. J Mol Biol, 2003. 326(3): p. 955-78.
16. Jambon, M., et al., A new bioinformatic approach to detect common 3D sites in protein structures. Proteins, 2003. 52(2): p. 137-45.
17. Yao, H., et al., An accurate, sensitive, and scalable method to identify functional sites in protein structures. J Mol Biol, 2003. 326(1): p. 255-61.
18. Bartlett, G. J., et al., Analysis of catalytic residues in enzyme active sites. J Mol Biol, 2002. 324(1): p. 105-21.
19. Gutteridge, A., G. J. Bartlett, and J. M. Thornton, Using a neural network and spatial clustering to predict the location of active sites in enzymes. J Mol Biol, 2003. 330(4): p. 719-34.
20. Ondrechen, M. J., J. G. Clifton, and D. Ringe, THEMATICS: a simple computational predictor of enzyme function from structure. Proc Natl Acad Sci USA, 2001. 98(22): p. 12473-8.
21. Elcock, A. H., Prediction of functionally important residues based solely on the computed energetics of protein structure. J Mol Biol, 2001. 312(4): p. 885-96.
22. DesJarlais, R. L., et al., Using shape complementarity as an initial screen in designing ligands for a receptor binding site of known three-dimensional structure. J Med Chem, 1988. 31(4): p. 722-9.
23. Laskowski, R. A., et al., Protein clefts in molecular recognition and function. Protein Sci, 1996. 5(12): p. 2438-52.
24. Ringe, D., What makes a binding site a binding site? Curr Opin Struct Biol, 1995. 5(6): p. 825-9.
25. Liang, J., H. Edelsbrunner, and C. Woodward, Anatomy of protein pockets and cavities: measurement of binding site geometry and implications for ligand design. Protein Sci, 1998. 7(9): p. 1884-97.
26. Hendlich, M., F. Rippmann, and G. Barnickel, LIGSITE: automatic and efficient detection of potential small molecule-binding sites in proteins. J Mol Graph Model, 1997. 15(6): p. 359-63, 389.
27. Peters, K. P., J. Fauck, and C. Frommel, The automatic search for ligand binding sites in proteins of known three-dimensional structure using only geometric criteria. J Mol Biol, 1996. 256(1): p. 201-13.
28. Brady, G. P., Jr. and P. F. Stouten, Fast prediction and visualization of protein binding pockets with PASS. J Comput Aided Mol Des, 2000. 14(4): p. 383-401.
29. Levitt, D. G. and L. J. Banaszak, POCKET: a computer graphics method for identifying and displaying protein cavities and their surrounding amino acids. J Mol Graph, 1992. 10(4): p. 229-34.
30. Laskowski, R. A., SURFNET: a program for visualizing molecular surfaces, cavities, and intermolecular interactions. J Mol Graph, 1995. 13(5): p. 323-30, 307-8.
31. Wodak, S. J. and J. Janin, Computer analysis of protein-protein interaction. J Mol Biol, 1978. 124(2): p. 323-42.
32. Fanning, D. W., J. A. Smith, and G. D. Rose, Molecular cartography of globular proteins with application to antigenic sites. Biopolymers, 1986. 25(5): p. 863-83.
33. Binkowski, T. A., S. Naghibzadeh, and J. Liang, CASTp: Computed Atlas of Surface Topography of proteins. Nucleic Acids Res, 2003. 31(13): p. 3352-5.
34. Silberstein, M., et al., Identification of substrate binding sites in enzymes by computational solvent mapping. J Mol Biol, 2003. 332(5): p. 1095-113.
35. Bhinge, A., et al., Accurate detection of protein:ligand binding sites using molecular dynamics simulations. Structure (Camb), 2004. 12(11): p. 1989-99.
36. Kundu, S., et al., Dynamics of proteins in crystals: comparison of experiment with simple models. Biophys J, 2002. 83(2): p. 723-32.
37. Schomaker, V. T., K. N, On the rigid-body motion of molecules in crystals. Acta Crystalog. sect. B, 1968. 34: p. 63-76.
38. Luque, I. and E. Freire, Structural stability of binding sites: consequences for binding affinity and allosteric effects. Proteins, 2000. Suppl 4: p. 63-71.
39. Amitai, G., et al., Network analysis of protein structures identifies functional residues. J Mol Biol, 2004. 344(4): p. 1135-46.
40. Fu, Z., et al., Crystal structure of glycine N-methyltransferase from rat liver. Biochemistry, 1996. 35(37): p. 11985-93.
41. Wilmanns, M., et al., Three-dimensional structure of the bifunctional enzyme phosphoribosylanthranilate isomerase: indoleglycerolphosphate synthase from Escherichia coli refined at 2.0 A resolution. J Mol Biol, 1992. 223(2): p. 477-507.
42. Miller, S., et al., Interior and surface of monomeric proteins. J Mol Biol, 1987. 196(3): p. 641-56.
43. Janin, J., S. Miller, and C. Chothia, Surface, subunit interfaces and interior of oligomeric proteins. J Mol Biol, 1988. 204(1): p. 155-64.
44. Nichols, B. P., Evolution of genes and enzymes of tryptophan biosynthesis. In: F. C. Neidhardt, Editor, Escherichia coli and Salmonella, 1996. 2 ASM Press, Washington, D.C.: p. 2638-2648.
45. Kim, H. and W. N. Lipscomb, X-ray crystallographic determination of the structure of bovine lens leucine aminopeptidase complexed with amastatin: formulation of a catalytic mechanismfeaturing a gem-diolate transition state. Biochemistry, 1993. 32(33): p. 8465-78.
46. Porter, C. T., G. J. Bartlett, and J. M. Thornton, The Catalytic Site Atlas: a resource of catalytic sites and residues identified in enzymes using structural data. Nucleic Acids Res, 2004. 32 Database issue: p. D129-33.
47. Orengo, C. A., et al., CATH—a hierarchic classification of protein domain structures. Structure, 1997. 5(8): p. 1093-108.
48. Pearl, F. M., et al., Assigning genomic sequences to CATH. Nucleic Acids Res, 2000. 28(1): p. 277-82.
49. Thunnissen, M. M., et al., Crystal structure of common type acylphosphatase from bovine testis. Structure, 1997. 5(1): p. 69-79.
50. West, A. H., E. Martinez-Hackert, and A. M. Stock, Crystal structure of the catalytic domain of the chemotaxis receptor methylesterase, CheB. J Mol Biol, 1995. 250(2): p. 276-90.
51. Djordjevic, S., et al., Structural basis for methylesterase CheB regulation by a phosphorylation-activated domain. Proc Natl Acad Sci USA, 1998. 95(4): p. 1381-6.
52. Strobl, S., et al., The crystal structure of calcium-free human m-calpain suggests an electrostatic switch mechanism for activation by calcium. Proc Natl Acad Sci USA, 2000. 97(2): p. 588-92.
53. Imajoh, S., et al., Molecular cloning of the cDNA for the large subunit of the high-Ca2+-requiring form of human Ca2+-activated neutral protease. Biochemistry, 1988. 27(21): p. 8122-8.
54. Kanaya, S., et al., Identification of the amino acid residues involved in an active site of Escherichia coli ribonuclease H by site-directed mutagenesis. J Biol Chem, 1990. 265(8): p. 4615-21.
55. Yang, W., et al., Structure of ribonuclease H phased at 2 A resolution by MA4D analysis of the selenomethionyl protein. Science, 1990. 249(4975): p. 1398-405.
56. Margaret A. Phillipsb, R. J. F., Proteases. Current Opinion in Structural Biology, 1992. 2: p. 713-720.
57. Ben-Zeev, E. and M. Eisenstein, Weighted geometric docking: incorporating external information in the rotation-translation scan. Proteins, 2003. 52(1): p. 24-7.
58. Ben-Zeev, E., et al., Prediction of the structure of the complex between the 30S ribosomal subunit and colicin E3 via weighted-geometric docking. J Biomol Struct Dyn, 2003. 20(5): p. 669-76.
59. Eisenstein, M., Geometric recognition as a tool for predicting structures of molecular complexes. Letters in Peptide Science, 1998. 5: p. 365-369.
60. Eisenstein, M., et al., Modeling supra-molecular helices: extension of the molecular surface recognition algorithm and application to the protein coat of the tobacco mosaic virus. J Mol Biol, 1997. 266(1): p. 13543.
61. Katchalski-Katzir, E., et al., Molecular surface recognition: determination of geometric fit between proteins and their ligands by correlation techniques. Proc Natl Acad Sci USA, 1992. 89(6): p. 2195-9.
62. Eisenstein, M. and E. Katchalski-Katzir, On proteins, grids, correlations, and docking. C R Biol, 2004. 327(5): p. 409-20.
63. Berchanski, A., B. Shapira, and M. Eisenstein, Hydrophobic complementarity in protein-protein docking. Proteins, 2004. 56(1): p. 130-42.
64. Heifetz, A., E. Katchalski-Katzir, and M. Eisenstein, Electrostatics in protein-protein docking. Protein Sci, 2002. 11(3): p. 571-87.
65. Engen, J. R., et al., Hydrogen exchange shows peptide binding stabilizes motions in Hck SH2. Biochemistry, 1999. 38(28): p. 8926-35.
66. Finucane, M. D. and O. Jardetzky, Mechanism of hydrogen-deuterium exchange in trp repressor studied by 1H-15N NMR. J Mol Biol, 1995. 253(4): p. 576-89.
67. McCallum, S. A., et al., Ligand-induced changes in the structure and dynamics of a human class Mu glutathione S-transferase. Biochemistry, 2000. 39(25): p. 7343-56.
68. Wang, F., J. S. Blanchard, and X. J. Tang, Hydrogen exchange/electrospray ionization mass spectrometry studies of substrate and inhibitor binding and conformational changes of Escherichia coli dihydrodipicolinate reductase. Biochemistry, 1997. 36(13): p. 3755-9.
69. Williams, D. C., Jr., et al., Global changes in amide hydrogen exchange rates for a protein antigen in complex with three different antibodies. J Mol Biol, 1996. 257(4): p. 866-76.
70. Jones, S. and J. M. Thornton, Searching for functional sites in protein structures. Curr Opin Chem Biol, 2004. 8(1): p. 3-7.
71. Thornton, J. M., et al., From structure to function: approaches and limitations. Nat Struct Biol, 2000. 7 Suppl: p. 9914.
72. Todd, A. E., C. A. Orengo, and J. M. Thornton, Evolution of protein function, from a structural perspective. Curr Opin Chem Biol, 1999. 3(5): p. 548-56.
73. Bairoch, A., The ENZYME data bank. Nucleic Acids Res, 1993. 21(13): p. 3155-6.
74. Nagano, N., C. T. Porter, and J. M. Thornton, The (betaalpha)(8) glycosidases: sequence and structure analyses suggest distant evolutionary relationships. Protein Eng, 2001. 14(11): p. 845-55.
75. Vita, C., Engineering novel proteins by transfer of active sites to natural scaffolds. Curr Opin Biotechnol, 1997. 8(4): p. 429-34.
76. Vita, Scorpion toxins as natural scaffolds for protein engineering. Proc Natl Acad Sci USA, 1995. 92(14): p. 6404-8.
77. Smith, J. W., K. Tachias, and E. L. Madison, Protein loop grafting to construct a variant of tissue-type plasminogen activator that binds platelet integrin alpha IIb beta 3. J Biol Chem, 1995. 270(51): p. 30486-90.
78. Wolfson, A. J., et al., Modularity of protein function: chimeric interleukin 1 beta s containing specific protease inhibitor loops retain function of both molecules. Biochemistry, 1993. 32(20): p. 5327-31.
79. Hynes, T. R., et al., Transfer of a beta-turn structure to a new protein context. Nature, 1989. 339(6219): p. 73-6.
80. Drakopoulou, E., et al., Changing the structural context of a functional beta-hairpin. Synthesis and characterization of a chimera containing the curaremimetic loop of a snake toxin in the scorpion alpha/beta scaffold. J Biol Chem, 1996. 271(20): p. 11979-87.
81. Haldane., J. B. S., Enzymes. Green. London., 1930.
82. Pauling, L., Nature, 1938. 161: p. 707.
83. Pauling, L., Chem. Eng. News, 1946. 24: p. 1375.
84. Britt, B. M., A shifting specificity model for enzyme catalysis. J Theor Biol, 1993. 164(2): p. 181-90.
85. B M., B., For enzymes, bigger is better. Biophys Chem, 1997. November 69(1): p. 63-70.
86. Harel, M., et al., Structure and evolution of the serum paraoxonase family of detoxifying and anti-atherosclerotic enzymes. Nat Struct Mol Biol, 2004. 11(5): p. 412-9.
87. Connolly, M. L., Solvent-accessible surfaces of proteins and nucleic acids. Science, 1983. 221(4612): p. 709-13.
88. Connolly, M. L., Analytical molecular surface calculation. J. Appl. Cryst, 1983. 16: p. 548:558.
89. Srere, P., Why are enzymes so big? Trends Biochem Sci, 1984. 9: p. 387-390.
90. Henrick, K. and J. M. Thornton, PQS: a protein quaternary structure file server. Trends Biochem Sci, 1998. 23(9): p. 358-61.

### Appendix

APPENDIX 1
Dataset-I.

| | | | Total | |
|---|---|---|---|---|
| | EC | PDB | surface | |
| | Number | code | dots | Rank |
| 1 | 1.1.1.21 | 1ADS | 86807 | 1 |
| 2 | 1.1.1.42 | 9ICD | 117619 | 1 |
| 3 | 1.1.1.44 | 1PGD | 141704 | 1 |
| 4 | 1.1.1.85 | 1IPD | 95361 | 1 |
| 5 | 1.1.3.15 | 1GOX | 98202 | 1 |
| 6 | 1.6.4.5 | 1TDE | 98639 | 1 |
| 7 | 1.6.6.1 | 1CND | 83194 | 1 |
| 8 | 1.6.99.1 | 1OYB | 105409 | 1 |
| 9 | 1.11.1.1 | 2NPX | 131476 | 1 |
| 10 | 1.11.1.5 | 1CCA | 85348 | 1 |
| 11 | 1.11.1.7 | 1ARP | 88742 | 1 |
| 12 | 1.14.13.2 | 1PBE | 113941 | 1 |
| 13 | 2.1.1.73 | 1HMY | 101194 | 1 |
| 14 | 2.3.1.28 | 3CLA | 68790 | 1 |
| 15 | 2.4.1.1 | 1GPB | 234726 | 1 |
| 16 | 2.4.2.1 | 1ULA | 89697 | 1 |
| 17 | 2.4.2.10 | 1STO | 66627 | 1 |
| 18 | 2.7.1.1 | 2YHX | 133015 | 1 |
| 19 | 2.7.2.3 | 1PHP | 108437 | 1 |
| 20 | 2.7.4.8 | 1GKY | 63828 | 1 |
| 21 | 3.1.1.3 | 1THG | 137278 | 1 |
| 22 | 3.1.1.7 | 1ACK | 126813 | 1 |
| 23 | 3.1.3.2 | 1RPA | 104336 | 1 |
| 24 | 3.1.26.4 | 1RNH | 50079 | 1 |
| 25 | 3.1.27.— | 1ONC | 35218 | 1 |
| 26 | 3.1.27.3 | 1FUT | 31824 | 1 |
| 27 | 3.1.27.5 | 1ROB | 39723 | 1 |
| 28 | 3.1.31.1 | 1SNC | 43962 | 1 |
| 29 | 3.2.1.2 | 1BYB | 128684 | 1 |
| 30 | 3.2.1.8 | 1XNB | 48640 | 1 |
| 31 | 3.2.1.73 | 1BYH | 57685 | 1 |
| 32 | 3.2.2.22 | 1FMP | 80344 | 1 |
| 33 | 3.4.17.1 | 2CTC | 76359 | 1 |
| 34 | 3.4.21.1 | 2GMT | 66099 | 1 |
| 35 | 3.4.21.4 | 1PPC | 59550 | 1 |
| 36 | 3.4.21.36 | 1ELA | 68082 | 1 |
| 37 | 3.4.21.37 | 1HNE | 63177 | 1 |
| 38 | 3.4.21.64 | 1PEK | 60997 | 1 |
| 39 | 3.4.21.80 | 3SGA | 42056 | 1 |
| 40 | 3.4.23.15 | 1SMR | 90821 | 1 |
| 41 | 3.4.23.20 | 1PPL | 80058 | 1 |
| 42 | 3.4.23.21 | 3APR | 86564 | 1 |
| 43 | 3.4.23.22 | 1EPM | 84996 | 1 |
| 44 | 3.4.23.23 | 1MPP | 87476 | 1 |
| 45 | 3.4.24.27 | 1HYT | 78197 | 1 |
| 46 | 3.4.24.46 | 1IAG | 58278 | 1 |
| 47 | 3.5.4.4 | 1ADD | 88013 | 1 |
| 48 | 3.8.1.5 | 2DHC | 82154 | 1 |
| 49 | 4.1.1.64 | 2DKB | 116358 | 1 |
| 50 | 4.1.3.7 | 1CSH | 126904 | 1 |
| 51 | 4.2.1.1 | 1CIL | 72828 | 1 |
| 52 | 4.2.1.3 | 8ACN | 177914 | 1 |
| 53 | 4.2.1.11 | 5ENL | 109510 | 1 |
| 54 | 4.2.99.18 | 1ABK | 66440 | 1 |
| 55 | 4.3.1.8 | 1PDA | 83718 | 1 |
| 56 | 5.1.2.2 | 1MNS | 90770 | 1 |
| 57 | 5.4.2.1 | 3PGM | 83960 | 1 |
| 58 | 6.3.4.15 | 1BIB | 82103 | 1 |
| 59 | 2.4.1.19 | 1CDG | 156939 | 2 |
| 60 | 3.2.1.18 | 2SIM | 94519 | 2 |
| 61 | 3.4.21.12 | 2ALP | 44888 | 2 |
| 62 | 3.4.22.2 | 1PIP | 60401 | 3 |
| 63 | 3.1.1.— | 2CUT | 49859 | 4 |
| 64 | 3.4.11.1 | 1BLL | 122581 | n.f |
| 65 | 4.1.1.47 | 1Pii | 123686 | n.f |

APPENDIX 2
Dataset-II.

| | | | | Number | | | | | | | |
|---|---|---|---|---|---|---|---|---|---|---|---|
| | | | Total | Of | Recognized Catalytic Residues/Atoms | | | | | | |
| | EC | PDB | Surface | Catalytic | Cluster 1 | | Cluster 2 | | Cluster3 | | |
| | Number | code | dots | residues | Residues | Atoms | Residues | Atoms | Residues | Atoms | Rank |
| 1 | 2.4.2.30 | 1A26 | 108601 | 2 | 1 | 9 | 0 | 0 | 0 | 0 | 1 |
| 2 | 4.2.1.96 | 1DCO | 32526 | 4 | 2 | 4 | 0 | 0 | 0 | 0 | 1 |
| 3 | 3.2.2.1 | 1MAS | 88107 | 3 | 3 | 14 | 0 | 0 | 0 | 0 | 1 |
| 4 | 2.7.3.3 | 1BG0 | 98993 | 5 | 5 | 18 | 1 | 1 | 0 | 0 | 1 |
| 5 | 3.5.1.1 | 3ECA | 90588 | 5 | 2 | 7 | 0 | 0 | 0 | 0 | 1 |
| 6 | 1.3.99.10 | 1IVH | 106841 | 1 | 1 | 2 | 1 | 1 | 0 | 0 | 1 |
| 7 | 4.6.1.1 | 1AB8 | 56584 | 1 | 0 | 0 | 0 | 0 | 0 | 0 | 1 |
| 8 | 3.1.3.2 | 1RPT | 106023 | 6 | 6 | 16 | 0 | 0 | 0 | 0 | 1 |
| 9 | 2.1.2.2 | 1GRC | 54653 | 4 | 3 | 11 | 3 | 3 | 0 | 0 | 1 |
| 10 | 2.4.1.1 | 1GPA | 237340 | 4 | 1 | 4 | 0 | 0 | 0 | 0 | 1 |
| 11 | 3.5.2.6 | 1BTL | 74856 | 4 | 3 | 6 | 0 | 0 | 0 | 0 | 1 |
| 12 | 2.7.2.3 | 13PK | 105336 | 4 | 4 | 12 | 0 | 0 | 0 | 0 | 1 |
| 13 | 2.7.4.6 | 1NSP | 47432 | 3 | 3 | 7 | 0 | 0 | 0 | 0 | 1 |
| 14 | 1.1.3.6 | 1COY | 122228 | 3 | 3 | 6 | 0 | 0 | 0 | 0 | 1 |
| 15 | 1.4.1.1 | 1PJB | 93549 | 4 | 4 | 10 | 0 | 0 | 0 | 0 | 1 |
| 16 | 3.5.1.11 | 1PNL | 179363 | 3 | 3 | 10 | 0 | 0 | 0 | 0 | 1 |
| 17 | 1.1.1.158 | 1MBB | 99753 | 3 | 3 | 9 | 2 | 4 | 0 | 0 | 1 |
| 18 | 4.6.1.13 | 2PLC | 78977 | 5 | 5 | 9 | 0 | 0 | 0 | 0 | 1 |
| 19 | 6.3.3.3 | 1DAE | 63534 | 4 | 4 | 13 | 0 | 0 | 0 | 0 | 1 |
| 20 | 4.1.2.13 | 1B57 | 96482 | 3 | 3 | 9 | 0 | 0 | 0 | 0 | 1 |
| 21 | 6.3.2.9 | 1UAG | 115007 | 3 | 3 | 12 | 0 | 0 | 0 | 0 | 1 |
| 22 | 1.11.1.11 | 1APX | 72792 | 3 | 2 | 10 | 0 | 0 | 2 | 2 | 1 |
| 23 | 2.4.2.1 | 1ULA | 89697 | 3 | 2 | 4 | 1 | 1 | 0 | 0 | 1 |
| 24 | 2.4.2.29 | 1PUD | 98146 | 1 | 1 | 3 | 0 | 0 | 0 | 0 | 1 |
| 25 | 1.11.1.10 | 1VNC | 150694 | 2 | 2 | 4 | 0 | 0 | 0 | 0 | 1 |
| 26 | 2.6.1.21 | 1DAA | 89086 | 3 | 3 | 10 | 1 | 1 | 0 | 0 | 1 |
| 27 | 1.1.1.34 | 1DQA | 119453 | 4 | 2 | 11 | 1 | 2 | 0 | 0 | 1 |
| 28 | 3.4.21.7 | 1BML | 70514 | 3 | 2 | 5 | 0 | 0 | 0 | 0 | 1 |
| 29 | 4.1.1.23 | 1DBT | 69467 | 2 | 2 | 9 | 0 | 0 | 0 | 0 | 1 |
| 30 | 1.2.1.8 | 1A4S | 136970 | 3 | 3 | 13 | 1 | 2 | 0 | 0 | 1 |
| 31 | 3.1.3.1 | 1ALK | 116415 | 2 | 2 | 7 | 0 | 0 | 0 | 0 | 1 |
| 32 | 1.1.1.38 | 1DO8 | 166565 | 3 | 2 | 10 | 0 | 0 | 0 | 0 | 1 |
| 33 | 3.2.1.10 | 1UOK | 147717 | 3 | 3 | 10 | 0 | 0 | 0 | 0 | 1 |
| 34 | 3.4.24.27 | 8TLN | 79491 | 2 | 2 | 5 | 0 | 0 | 0 | 0 | 1 |
| 35 | 1.5.3.1 | 1B3M | 100442 | 4 | 4 | 12 | 0 | 0 | 0 | 0 | 1 |
| 36 | 3.1.21.2 | 1QUM | 73653 | 1 | 1 | 8 | 0 | 0 | 0 | 0 | 1 |
| 37 | 1.1.1.22 | 1DLI | 122326 | 6 | 5 | 12 | 1 | 1 | 0 | 0 | 1 |
| 38 | 4.2.2.10 | 1IDJ | 86022 | 2 | 2 | 8 | 0 | 0 | 0 | 0 | 1 |
| 39 | 1.11.1.6 | 1IPH | 227781 | 3 | 3 | 12 | 0 | 0 | 0 | 0 | 1 |
| 40 | 1.5.1.5 | 1A4I | 83485 | 1 | 1 | 3 | 0 | 0 | 0 | 0 | 1 |
| 41 | 5.3.1.1 | 1TPH | 40169 | 5 | 2 | 7 | 2 | 3 | 0 | 0 | 1 |
| 42 | 1.17.99.1 | 1DII | 145071 | 6 | 3 | 14 | 0 | 0 | 0 | 0 | 1 |
| 43 | 3.1.3.11 | 1EYI | 94019 | 3 | 3 | 3 | 0 | 0 | 0 | 0 | 1 |
| 44 | 2.1.3.3 | 1AKM | 85246 | 6 | 6 | 22 | 0 | 0 | 0 | 0 | 1 |
| 45 | 2.1.4.1 | 1JDW | 92561 | 7 | 7 | 16 | 2 | 3 | 0 | 0 | 1 |
| 46 | 3.2.1.113 | 1DL2 | 123529 | 4 | 4 | 12 | 0 | 0 | 0 | 0 | 1 |
| 47 | 3.2.2.21 | 1DIZ | 84254 | 3 | 2 | 8 | 0 | 0 | 0 | 0 | 1 |
| 48 | 3.5.2.6 | 2BMI | 62566 | 2 | 1 | 5 | 0 | 0 | 0 | 0 | 1 |
| 49 | 6.3.4.4 | 1GIM | 116223 | 3 | 3 | 16 | 0 | 0 | 0 | 0 | 1 |
| 50 | 3.4.24.17 | 1HFS | 52056 | 2 | 1 | 2 | 0 | 0 | 0 | 0 | 1 |
| 51 | 5.4.2.9 | 1PYM | 96779 | 4 | 3 | 6 | 0 | 0 | 0 | 0 | 1 |
| 52 | 1.1.1.2 | 2ALR | 87739 | 2 | 2 | 4 | 0 | 0 | 1 | 1 | 1 |
| 53 | 3.1.3.33 | 1D8H | 92557 | 4 | 3 | 6 | 0 | 0 | 0 | 0 | 1 |
| 54 | 4.2.1.70 | 1DJ0 | 80130 | 1 | 1 | 6 | 0 | 0 | 0 | 0 | 1 |
| 55 | 1.1.99.28 | 1OFG | 110023 | 2 | 2 | 11 | 0 | 0 | 0 | 0 | 1 |
| 56 | 2.3.1.39 | 1MLA | 77536 | 3 | 2 | 5 | 0 | 0 | 0 | 0 | 1 |
| 57 | 3.1.4.3 | 1AH7 | 66885 | 1 | 1 | 4 | 0 | 0 | 0 | 0 | 1 |
| 58 | 3.4.22.16 | 8PCH | 60276 | 4 | 4 | 12 | 2 | 4 | 0 | 0 | 1 |
| 59 | 1.8.1.7 | 1GET | 136829 | 7 | 5 | 27 | 0 | 0 | 0 | 0 | 1 |
| 60 | 3.1.30.2 | 1SMN | 64959 | 3 | 3 | 10 | 0 | 0 | 0 | 0 | 1 |
| 61 | 5.1.1.3 | 1B73 | 75570 | 6 | 5 | 13 | 0 | 0 | 0 | 0 | 1 |
| 62 | 1.1.1.25 | 1DQS | 103188 | 1 | 1 | 2 | 0 | 0 | 0 | 0 | 1 |
| 63 | 3.6.1.29 | 5FIT | 42928 | 3 | 2 | 8 | 0 | 0 | 0 | 0 | 1 |
| 64 | 3.4.21.92 | 1TYF | 63628 | 5 | 4 | 16 | 0 | 0 | 0 | 0 | 1 |
| 65 | 4.2.3.7 | 1PS1 | 85915 | 8 | 5 | 13 | 0 | 0 | 0 | 0 | 1 |
| 66 | 2.7.1.38 | 2PHK | 92012 | 2 | 2 | 4 | 2 | 5 | 0 | 0 | 1 |
| 67 | 4.1.1.39 | 1RBL | 125478 | 6 | 3 | 7 | 0 | 0 | 0 | 0 | 1 |
| 68 | 3.5.3.3 | 1CHM | 110021 | 3 | 3 | 7 | 0 | 0 | 0 | 0 | 1 |
| 69 | 3.3.1.1 | 1B3R | 116447 | 5 | 5 | 15 | 0 | 0 | 0 | 0 | 1 |
| 70 | 1.13.11.2 | 1MPY | 86460 | 3 | 3 | 8 | 0 | 0 | 0 | 0 | 1 |
| 71 | 2.3.3.9 | 1D8C | 188832 | 4 | 3 | 8 | 0 | 0 | 0 | 0 | 1 |
| 72 | 2.3.3.1 | 1AL6 | 123498 | 3 | 3 | 12 | 0 | 0 | 0 | 0 | 1 |
| 73 | 4.1.1.49 | 1AQ2 | 140824 | 3 | 3 | 8 | 1 | 2 | 0 | 0 | 1 |
| 74 | 1.5.1.3 | 1RA2 | 51989 | 7 | 6 | 21 | 0 | 0 | 0 | 0 | 1 |
| 75 | 3.2.1.132 | 1CHK | 67079 | 2 | 1 | 7 | 0 | 0 | 0 | 0 | 1 |
| 76 | 6.3.1.1 | 12AS | 93423 | 3 | 3 | 8 | 0 | 0 | 0 | 0 | 1 |
| 77 | 2.4.1.27 | 1C3J | 102861 | 2 | 2 | 9 | 0 | 0 | 0 | 0 | 1 |
| 78 | 3.5.1.28 | 1LBA | 44880 | 2 | 2 | 7 | 0 | 0 | 0 | 0 | 1 |
| 79 | 3.2.2.3 | 1EUG | 65750 | 2 | 2 | 6 | 0 | 0 | 0 | 0 | 1 |
| 80 | 5.4.99.2 | 1REQ | 203788 | 5 | 3 | 21 | 0 | 0 | 0 | 0 | 1 |
| 81 | 2.4.2.8 | 1BZY | 67837 | 5 | 4 | 9 | 0 | 0 | 0 | 0 | 1 |
| 82 | 1.1.1.3 | 1EBF | 106933 | 2 | 2 | 8 | 0 | 0 | 0 | 0 | 1 |
| 83 | 1.11.1.10 | 2CPO | 87824 | 2 | 2 | 10 | 0 | 0 | 0 | 0 | 1 |
| 84 | 2.1.1.20 | 1XVA | 95220 | 1 | 0 | 0 | 0 | 0 | 0 | 0 | 1 |
| 85 | 2.7.1.11 | 1PFK | 92319 | 5 | 5 | 17 | 0 | 0 | 0 | 0 | 1 |
| 86 | 2.4.2.21 | 1D0S | 82672 | 1 | 1 | 2 | 0 | 0 | 0 | 0 | 1 |
| 87 | 2.5.1.7 | 1UAE | 102967 | 4 | 4 | 16 | 0 | 0 | 0 | 0 | 1 |
| 88 | 3.5.1.59 | 1NBA | 77151 | 5 | 4 | 7 | 0 | 0 | 0 | 0 | 1 |
| 89 | 3.5.99.6 | 1CD5 | 77138 | 4 | 2 | 7 | 0 | 0 | 0 | 0 | 1 |
| 90 | 2.1.1.45 | 1LCB | 99277 | 6 | 4 | 9 | 0 | 0 | 0 | 0 | 1 |
| 91 | 5.1.3.13 | 1DZR | 61029 | 2 | 1 | 4 | 0 | 0 | 0 | 0 | 1 |
| 92 | 3.5.1.26 | 1APY | 47605 | 4 | 3 | 11 | 0 | 0 | 0 | 0 | 1 |
| 93 | 1.2.1.11 | 1BRM | 104803 | 3 | 2 | 5 | 0 | 0 | 0 | 0 | 1 |
| 94 | 6.3.2.3 | 2HGS | 130408 | 4 | 4 | 15 | 0 | 0 | 0 | 0 | 1 |
| 95 | 3.4.23.23 | 1MPP | 87476 | 4 | 4 | 12 | 0 | 0 | 0 | 0 | 1 |
| 96 | 2.7.1.69 | 1GPR | 45929 | 4 | 2 | 4 | 0 | 0 | 0 | 0 | 1 |
| 97 | 4.2.1.60 | 1MKA | 53710 | 5 | 2 | 5 | 0 | 0 | 0 | 0 | 1 |
| 98 | 3.1.27.1 | 1BOL | 62750 | 3 | 3 | 9 | 0 | 0 | 0 | 0 | 1 |
| 99 | 1.3.99.1 | 1D4C | 159275 | 4 | 2 | 6 | 0 | 0 | 0 | 0 | 1 |
| 100 | 1.2.7.1 | 2PDA | 246963 | 4 | 3 | 10 | 0 | 0 | 0 | 0 | 1 |
| 101 | 3.6.1.1 | 1WGI | 82101 | 1 | 1 | 4 | 0 | 0 | 0 | 0 | 1 |
| 102 | 1.1.1.35 | 2HDH | 93131 | 4 | 3 | 8 | 2 | 3 | 0 | 0 | 1 |
| 103 | 4.2.1.10 | 1QFE | 70462 | 3 | 3 | 5 | 0 | 0 | 0 | 0 | 1 |
| 104 | 2.5.1.2 | 2THI | 105084 | 2 | 2 | 3 | 0 | 0 | 0 | 0 | 1 |
| 105 | 4.2.2.5 | 1CB8 | 173601 | 3 | 2 | 8 | 0 | 0 | 0 | 0 | 1 |
| 106 | 1.13.11.12 | 1LNH | 230298 | 1 | 0 | 0 | 0 | 0 | 0 | 0 | 1 |
| 107 | 5.3.4.1 | 1MEK | 47053 | 4 | 0 | 0 | 0 | 0 | 0 | 0 | 1 |
| 108 | 3.1.1.4 | 1AE7 | 41801 | 3 | 2 | 6 | 1 | 3 | 0 | 0 | 1 |
| 109 | 2.7.7.48 | 1CQQ | 51259 | 4 | 4 | 16 | 0 | 0 | 0 | 0 | 1 |
| 110 | 4.2.1.3 | 1FGH | 180132 | 7 | 5 | 14 | 2 | 5 | 0 | 0 | 1 |
| 111 | 1.4.99.3 | 2BBK | 38445 | 6 | 4 | 13 | 0 | 0 | 0 | 0 | 1 |
| 112 | 4.4.1.5 | 1FRO | 65796 | 1 | 1 | 1 | 0 | 0 | 0 | 0 | 1 |
| 113 | 3.1.21.1 | 1DNK | 63312 | 4 | 3 | 6 | 0 | 0 | 0 | 0 | 1 |
| 114 | 3.1.1.47 | 1BWP | 63761 | 5 | 4 | 9 | 0 | 0 | 0 | 0 | 1 |
| 115 | 2.7.1.40 | 1PKN | 154984 | 6 | 5 | 13 | 0 | 0 | 0 | 0 | 1 |
| 116 | 1.8.1.2 | 1AOP | 124047 | 5 | 4 | 12 | 0 | 0 | 0 | 0 | 1 |
| 117 | 2.3.1.87 | 1B6B | 54892 | 5 | 3 | 12 | 0 | 0 | 0 | 0 | 1 |
| 118 | 2.6.1.16 | 1MOQ | 96881 | 5 | 2 | 2 | 0 | 0 | 0 | 0 | 1 |
| 119 | 1.1.3.38 | 1VAO | 151829 | 5 | 5 | 15 | 1 | 2 | 0 | 0 | 1 |
| 120 | 4.2.1.47 | 1DB3 | 97304 | 4 | 4 | 8 | 0 | 0 | 0 | 0 | 1 |
| 121 | 2.3.1.16 | 1AFW | 94846 | 4 | 4 | 9 | 0 | 0 | 0 | 0 | 1 |
| 122 | 4.1.99.3 | 1DNP | 138383 | 3 | 1 | 2 | 0 | 0 | 0 | 0 | 1 |
| 123 | 4.2.1.11 | 5ENL | 109510 | 4 | 4 | 13 | 1 | 2 | 0 | 0 | 1 |
| 124 | 1.1.1.85 | 1A05 | 99676 | 3 | 1 | 2 | 0 | 0 | 0 | 0 | 1 |
| 125 | 2.7.4.3 | 1ZIO | 67781 | 6 | 4 | 9 | 0 | 0 | 0 | 0 | 1 |
| 126 | 2.4.2.2 | 1BRW | 119796 | 4 | 2 | 5 | 0 | 0 | 0 | 0 | 1 |
| 127 | 3.5.1.88 | 1BS4 | 55373 | 4 | 4 | 11 | 0 | 0 | 1 | 2 | 1 |
| 128 | 1.11.1.7 | 1MHL | 147663 | 1 | 1 | 8 | 0 | 0 | | 0 | 1 |
| 129 | 3.4.16.6 | 1BCR | 85239 | 3 | 3 | 8 | 1 | 1 | 0 | 0 | 1 |
| 130 | 4.2.1.84 | 1AHJ | 77354 | 1 | 1 | 9 | 0 | 0 | 0 | 0 | 1 |
| 131 | 1.14.99.1 | 5COX | 161373 | 4 | 1 | 2 | 3 | 11 | 0 | 0 | 2 |
| 132 | 4.1.1.41 | 1EF8 | 82537 | 3 | 0 | 0 | 3 | 8 | 0 | 0 | 2 |
| 133 | 5.4.99.5 | 3CSM | 82889 | 4 | 0 | 0 | 1 | 2 | 0 | 0 | 2 |
| 134 | 4.1.2.17 | 1FUA | 64534 | 2 | 0 | 0 | 1 | 2 | 0 | 0 | 2 |
| 135 | 4.2.3.3 | 1B93 | 47767 | 6 | 0 | 0 | 1 | 2 | 0 | 0 | 2 |
| 136 | 3.8.1.2 | 1QQ5 | 67831 | 9 | 3 | 12 | 6 | 9 | 1 | 1 | 2 |
| 137 | 3.4.22.2 | 9PAP | 59359 | 4 | 1 | 2 | 3 | 7 | 0 | 0 | 2 |
| 138 | 3.1.3.48 | 1YTW | 79586 | 6 | 0 | 0 | 3 | 7 | 0 | 0 | 2 |
| 139 | 2.1.4.2 | 1BWD | 98894 | 7 | 2 | 10 | 6 | 15 | 0 | 0 | 2 |
| 140 | 1.14.13.25 | 1MHY | 149410 | 2 | 0 | 0 | 1 | 2 | 0 | 0 | 2 |
| 141 | 1.15.1.1 | 2JCW | 43768 | 2 | 0 | 0 | 2 | 7 | 0 | 0 | 2 |
| 142 | 2.1.1.63 | 1ADN | 45678 | 1 | 1 | 1 | 1 | 2 | 0 | 0 | 2 |
| 143 | 2.3.1.41 | 1KAS | 104343 | 4 | 1 | 4 | 4 | 10 | 0 | 0 | 2 |
| 144 | 2.3.1.54 | 2PFL | 185468 | 4 | 0 | 0 | 2 | 4 | 0 | 0 | 2 |
| 145 | 3.5.4.16 | 1GTP | 77865 | 2 | 0 | 0 | 0 | 0 | 1 | 1 | 3 |
| 146 | 1.1.99.8 | 1G72 | 131981 | 1 | 0 | 0 | 0 | 0 | 0 | 0 | 3 |
| 147 | 3.5.1.52 | 1PGS | 84365 | 2 | 0 | 0 | 0 | 0 | 0 | 0 | 3 |
| 148 | 3.5.4.5 | 1CTT | 76891 | 1 | 0 | 0 | 0 | 0 | 1 | 2 | 3 |
| 149 | 4.1.1.22 | 1PYA | 77165 | 2 | 1 | 3 | 0 | 0 | 2 | 3 | 3 |
| 150 | 2.7.7.12 | 1HXQ | 103195 | 7 | 1 | 4 | 0 | 0 | 0 | 0 | 4 |
| 151 | 2.4.2.19 | 1QPR | 80185 | 4 | 0 | 0 | 1 | 4 | 0 | 0 | 4 |
| 152 | 2.7.1.69 | 1E2A | 35901 | 4 | 1 | 1 | 1 | 1 | 0 | 0 | 4 |
| 153 | 1.1.3.9 | 1GOG | 145952 | 4 | 1 | 2 | 0 | 0 | 0 | 0 | n.f |
| 154 | 3.8.1.6 | 1NZY | 87179 | 5 | 0 | 0 | 0 | 0 | 0 | 0 | n.f |
| 155 | 4.1.1.11 | 1AW8 | 35383 | 1 | 0 | 0 | 0 | 0 | 0 | 0 | n.f |
| 156 | 3.1.1.61 | 1CHD | 53162 | 5 | 0 | 0 | 0 | 0 | 0 | 0 | n.f |
| 157 | 3.6.1.7 | 2ACY | 32106 | 2 | 0 | 0 | 0 | 0 | 0 | 0 | n.f |
| 158 | 3.1.3.2 | 4KBP | 106439 | 3 | 0 | 0 | 0 | 0 | 0 | 0 | n.f |
| 159 | 2.3.1.129 | 1LXA | 70882 | 1 | 0 | 0 | 0 | 0 | 0 | 0 | n.f |
| 160 | 4.2.1.104 | 1DW9 | 62047 | 2 | 0 | 0 | 0 | 0 | 0 | 0 | n.f |
| 161 | 4.2.3.12 | 1B66 | 47002 | 4 | 0 | 0 | 0 | 0 | 0 | 0 | n.f |
| 162 | 1.7.3.3 | 1UOX | 103567 | 2 | 0 | 0 | 0 | 0 | 0 | 0 | n.f |
| 163 | 1.13.11.3 | 3PCA | 84268 | 2 | 1 | 1 | 0 | 0 | 0 | 0 | n.f |
| 164 | 5.3.2.— | 1BJP | 25573 | 3 | 0 | 0 | 0 | 0 | 0 | 0 | n.f |
| 165 | 5.99.1.2 | 1I7D | 191612 | 4 | 1 | 1 | 0 | 0 | 0 | 0 | n.f |
| 166 | 2.1.1.72 | 2ADM | 109458 | 3 | 0 | 0 | 0 | 0 | 0 | 0 | n.f |
| 167 | 5.3.1.25 | 1FUI | 160895 | 2 | 0 | 0 | 0 | 0 | 0 | 0 | n.f |
| 168 | 1.7.2.1 | 1NID | 100060 | 2 | 0 | 0 | 0 | 0 | 0 | 0 | n.f |
| 169 | 3.5.1.5 | 1KRB | 138091 | 4 | 0 | 0 | 0 | 0 | 0 | 0 | n.f |
| 170 | 1.14.13.7 | 1FOH | 176875 | 3 | 0 | 0 | 0 | 0 | 0 | 0 | n.f |
| 171 | 4.3.2.2 | 1C3C | 129687 | 2 | 0 | 0 | 0 | 0 | 0 | 0 | n.f |
| 172 | 1.6.99.2 | 1D4A | 85847 | 3 | 0 | 0 | 0 | 0 | 0 | 0 | n.f |
| 173 | 3.4.22.17 | 1KFU | 229897 | 4 | 0 | 0 | 0 | 0 | 0 | 0 | n.f |
| 174 | 2.5.1.6 | 1FUG | 117623 | 7 | 0 | 0 | 1 | 1 | 1 | 1 | n.f |
| 175 | 6.3.5.2 | 1GPM | 146698 | 5 | 0 | 0 | 0 | 0 | 0 | 0 | n.f |
| 176 | 6.5.1.2 | 1DGS | 185626 | 4 | 0 | 0 | 0 | 0 | 0 | 0 | n.f |

APPENDIX 3
List of Biosym van der Waals radii.

| | Atom | Description | Radii |
|---|---|---|---|
| 1 | C2 | CH2 group | 1.9 |
| 2 | C3 | CH3 group | 1.95 |
| 3 | CO | Aldehyde C | 1.8 |
| 4 | COa | Carboxylic/amide C | 1.8 |
| 5 | CH | Aromatic CH group | 1.9 |
| 6 | Car | Aromatic C (no H) | 1.8 |
| 7 | OH | Hydroxyl O | 1.7 |
| 8 | OHp | Phenolic OH | 1.7 |
| 9 | OHa | Carboxylic OH | 1.6 |
| 10 | OC | Aldehyde O | 1.6 |
| 11 | OCa | Carboxylic carbonyl | 1.6 |
| 12 | OCo | COOH carbonyl O | 1.6 |
| 13 | OP | Phosphate O | 1.64 |
| 14 | NH | Amine N | 1.7 |
| 15 | NH+ | Protonated Amine N | 1.75 |
| 16 | NA | Amide N | 1.65 |
| 17 | Nn | Nucleotide aromatic | 1.55 |
| 18 | NO | Nitro N | 1.65 |
| 19 | PO | Phosphate P | 1.8 |
| 20 | SH | Thiol & thioether S | 1.9 |

What is claimed is:

1. A method for determining the location of the active site of an enzyme, comprising:
determining a location of a reference point inside a functional unit of the enzyme;
determining a limiting distance from said reference point; and
identifying one or more molecular surface portions within said limiting distance to determine whether said molecular surface portion is part of the active site.

2. The method of claim 1, wherein said limiting distance is determined according to a minimum threshold.

3. The method of claim 2, wherein said minimum threshold is determined by comparing a distance of a plurality of surface dots, residues or atoms of the enzyme to said reference point.

4. The method of claim 1, wherein said comparing said distance to said reference point is performed for at least a portion of the atoms of the enzyme, including those of the core.

5. The method of claim 1, wherein said reference point comprises a point selected from the group consisting of a centroid, a center of mass, graph defined centrality or any other center.

6. The method of claim 1, wherein said functional unit is selected from the group consisting of a monomer, domain, chain, monomers, domains, chains or an entirety of the enzyme.

7. The method of claim 1, wherein said molecular surface portion or portions comprise one or more amino acid residues or atoms.

8. The method of claim 1, further comprising:
detecting a plurality of surface portions; and
ranking said surface portions by size to determine the active site.

9. The method of claim 1, further comprising:
detecting a plurality of surface portions; and
ranking said surface portions by distance to determine the active site.

10. The method of claim 1, further comprising:
receiving a plurality of surface portions from an external source; and
ranking said surface portions by distance and/or by size to determine the active site.

11. The method of claim 1, further comprising:
searching for a plurality of surface portions which reside in close proximity to a reference point of the molecule.

12. The method of claim 11, wherein a number of said plurality of surface portions is limited according to a threshold.

13. The method of claim 1, wherein the enzyme features an active site selected from the group consisting of buried or flat and shallow active sites.

14. The method of claim 1, wherein said comparing comprises selecting at least one putative active site group of amino acid residues from an internal group of amino acids according to Mcluster, wherein Mcluster is the minimum surface area in an internal cluster that renders it a putative active site cluster.

15. The method of claim 1, wherein said reference point comprises a centroid and wherein said comparing comprises:
clustering surface dots, atoms or amino acid residues according to a distance from said centroid; and
selecting surface accessible clusters.

16. The method of claim 15, wherein said selecting surface accessible clusters is performed according to a maximum surface area of said clusters.

17. The method of claim 16, wherein said selecting comprises:
selecting a surface accessible cluster having a surface area smaller than a maximum surface area; and
extending said surface accessible cluster to include a second cluster, wherein said surface accessible cluster is rejected if said combined cluster represents an internal void.

18. The method of claim 1, wherein said reference center comprises a center of mass.

19. The method of claim 18, wherein said identifying further comprises:
selecting the surface portion according to size and increasing proximity to said center of mass.

20. The method of claim 1, wherein said determining said location of a reference point comprises determining said location of said reference point for a plurality of functional units, such that the location is determined for a plurality of putative active sites;
ranking said plurality of putative active sites according to at least one parameter; and
selecting said active site from said plurality putative active sites according to said ranking.

21. The method of claim 20, wherein said at least one parameter is determined according to at least one characteristic of said functional unit.

22. The method of claim 21, wherein said at least one characteristic includes at least one of a biological unit and a domain classification.

23. The method of claim 1, further comprising:
providing a plurality of potential functional units; and
selecting a functional unit from said plurality of potential functional units according to a location of said active site.

24. The method of claim 23, wherein said determining said location of a reference point comprises determining said location of said reference point for said plurality of functional units, such that the location is determined for a plurality of putative active sites;
ranking said plurality of putative active sites according to at least one parameter; and
selecting said active site from said plurality of putative active sites according to said ranking.

25. The method of claim 24, wherein said at least one parameter is determined according to at least one characteristic of said putative functional unit.

26. The method of claim 25, wherein said at least one characteristic includes at least one of a biological unit and a domain classification.

27. The method of claim 23, wherein said at least one parameter is determined according to formation of continuous sites.

28. The method of claim 20, wherein said at least one parameter is determined according to formation of continuous sites.

29. The method of claim 23, wherein said functional unit is selected according to said active site.

30. A method for determining docking of a ligand to an enzyme, comprising:
providing a plurality of docking solutions for docking the ligand to the enzyme;
determining a limiting distance between the ligand docking site and a reference point of the enzyme; and
ranking said docking solutions at least according to said limiting distance.
